SEQUENCE LISTING (20 sequences)

SEQ ID NO: 1 - DNA, 1842 bp, Artificial
  recombinant sequence
ctggttccgcgtggatccatcaaagacgtgaaatcagataacaaaaccccgctggatctgcgtgccagctctccgaacatcatcaaagaatcagaaaaagtcaaatacgtggcgctgggcgactctattagtgccggttttgattcgcagctggatcaagactatccgggcaattacgataaagagaaaaacgaagtgaccggtatttctttcccgacgtacctggcatctttcatcaaaaacagtaacgctgataactctaaactggaaagttacaccaactacgcgagttccggcaccacgctggaagattggaaattcctgctgacggtggatgacagcaaactgaaacaactggcggaaaacgacaaaaacaaactggcctactacaaatctaaattcggtaaagatatcatcaaaaaagccaaagaagtgcgtgaagaactgcagaaagccaatctggttaccattacgctggcggccaacgatttcatggaactggtcgccaaaaaactgaaaaacccggaattcatcaaacagatccaaaacatcaaagcaaacccgaccaatgcgattgccggcgtttcatcggctttccaggatacgttcaaagaaatgaaaaaacgcatcgatgacttcagcgcagaactgaaaaaacatgtgaaaaccgaaaacgttaacttcatcggttacccgctgccgctgccgcacctgttcgtgctgctggatggctacctgtttaaaaacaatggtagctctctgatcgtctcacagatggcggtggatgccctgaataaaaacattcagtcgcaagttaccaaaaacaaatttaacttcatcaacccgtacgacttctcgttctggaccgataatagcaacattaataaactgtctccgacgctgttcgacatccatccgggcagttttggttacaagaaaatggcacaagatgttttcatcaaactgattgtcccggaacgtaatagcgacaaactgaacgaaaacggcatcaaatgggataacaaatacatcttcaccgataaagactcattcaccacgcagctggaattcgcgaacaaatacgaaaccgttaaaaaagtcctgacggatgacattaacggtaacctgtttaaagaagacgaaatcttcaaaaaattcagcgataaacgtaaaaacaaatgggaatggtacaaaaaacgcgtgctgaacagttccctggttaaaaatattctgaacggtgcaatcgacagtctgattcagtttccgaccatcaaagaagctgatctgtcatcgcgcctgtccaactttttcacgaaaaacaacaacgaaaacctgaacaaactgaaaaaatggctggaagaaagcgaatttgtgccgaaattcctgctgagatctgaactcgagtttcaggaaacctactggaacaaagacaaaaaatcttctagtgaacgcgcgaaactgttcgatctgatcaacatcttcaaaaacaacctgctgaacgaagcaaacctgatctccctgattgcttcatttttctccagcgatctgatccaaaaagaaaaagaagaactgaaagaaatcatcgcagaattcgctcagaacctgatcatcaaaaacgtggacgaaaaaatgctgagcgatgcaatcaataaagctattggcaataattcgagcaaagcgttcaaaaccgaaaacctggccaaactgatcaaaagtgttctgtctagtaaatccattaagaaaaccattggcgaactgttcatcaacctggcgaacaacagtaaaatctatggtcaggcgaaatccgtggtcgactcgagcggccgcatcgtgactgactga

SEQ ID NO: 2 - DNA, 1110 bp, Artificial
  recombinant sequence
ctggttccgcgtggatccgaaaatggtacaattaaggcaaaagaagttgcagaagtgcatcttccagataatttcgatataaaaaaacttaattcttctgaaaaaagagtttttaaaaaggcaaattctgtaaatttaaatagtgtagattatacaaaaggaactacctttgaaatacctggtgaaaaagattactttttaaaaatttattctttaactccaccatcaattgatgaaaatacagttgtgcctcttgataaggataaagaagttagtgctgaaataaatttggaatttaaaatcatttacaaagaccctgttgaaaacactgttgagttagtagatggctgggagaaaaaacttagcattaaaaatgacaaatttattttacattcgcttaattcagtttataacagtttcgaaagtaacaaagccagtgcttttgaatttaaaaacatagattgggacaaattgaaggctaaatattttgatgcagaaatagttaaatggagcgatggtgatacacctgtagttaaagttactagtgttgatcctttaaatcagaatgttaaagtaggtgatgaaactaaaataagaatttctggcgttgatacacctgaaaaatttgtgggtggaattaaaagcagtaatctagaacacaactatgcattactttcatctaaatttgctgaagaagctttaccaaagggctcaaaagttagagtttatggcgattctaaagatgcatacggaagaatagttggcgatgtattttttggagacaaatatcaatatctatattcagttgaaataactcgttctggattaactttaccatattatgacaatgtaactttggcatttagttttgtaaaaaatccagtttactgggagcattatgccttgcttcgtttaggtgaagcattagaatatgctaaaaccaacaaaaaaggattctttaaaacatttgcattgccatactggattcaatctaacatttacaaaatgaagcctaatggaaaatggacattgctagataaacactctcctacaaatctgtacaacattgaaggcattaatttaattgatgattcatacaaaaataaataa

SEQ ID NO: 3 - DNA, 2172 bp, Artificial
  recombinant sequence
ctggttccgcgtggatccaatgatcctaaaaataattctgaagttatcatatatcaacgtaatgctaattctgataaaacgactgaacaatttgaaaaatataatcaagttaacttactttcaaatataaaagattactttgataaacatgaacatttaaatttaatatcatataagggagggggcaagcctgagacagttgaatatagtttaatgatgcaaaacaactatatgagtaagtacatcaattttgatactgaagctttcaaggcaattgttaaaaaagaactaaaactaagcgacaagttgctaagtaggcttaaattctcatatgactataacaacgttacacgtgacccaggaaataattatgatgttctattcaaggttaaagttggccttccgctagaatcaaatgacaaaagcaaatatgaaagtggcttatacagccaacaaatcatagactttagaatcaaaaatgtaaaagtcaaagataatgataaaccttttgctgatgcattgaagccttatgatcaaaaaattcaagctttaactagtcaagactttgaagtcaagctagaaaatattgatgatgaattaaaacaagctattacaaaatatggcattcatcaactaagttcgaagcagtatgaaaagttattaaaattttctagtgctaaattagatgaaatattaaaagacaatgacaaagaattagaaatacctgttggtagcaaaaaagaaaaatatacgcttaaattcaataaattcattgaagatgttgtgctaaatgatggctcactatcaaatgctaaagcaaaaattagaattgctgcttcattgtttgacagcaagaaaaagtctactgtttgggaatcaggaaaaacagtttatggcttatttacagtacctaaagagcaaaaattaattagcgacttgcagttagctgaattaattgatgtacacacaattggtaacattgagcacaatagtgatctgtcaactttaacaaaaggcaacttatttattaacgtaaaagatgcaaatattgaaaaagttgaaattgataaaattacaactcaagactttagaaatgcaactgttactttaaatgttaaacttaaaaactcgcaggaattagttaaagttgaaaaacatcttggtgtaggtagatactcattgctttttgataaacaatttacaaaacaaaatattaaagctcctgcttttgctactgaaggaataacaactaaaaacttaccaagtattgataaaaccttttttggtcactataattcacaactattttcaggcggatatgcttcagcaagagctttctatgctgataatgtagttgagcccaaatatgtgcacattggtgaagactatattgcaccagattttcaaccagtcttggctccttatgacggacaaattgttgctgcttatgaacttacaactaaggtggtggctactggtgttggtactgttgttgttattaaaattcctgttgctaatttagattggagtccaaaagaaaaagaaatctatcttaatggcaacaatgaacatatttatatgtcattcttgcacttagatgccggtaaaaccttaaataacactagtttaggctggcaatcagaaacagtaacattgggcaagagaacaattaaggttgtcccaactgtgtctgctgatacacctactgaggtcaaaaaaggccaaattataggctttttgggaactcaggacactaatggtggatggatggcacatgcccatgttaacttatacacaaacagaaatttctggctttctccaaatcattttaacaaggaatcaaaacaatcaaacattgacaaaagagttaaggagtataaaaaaactgataagaataaaaaaataacatacagaattgtcggcaatgctggtgttgaagtttttttaaaccctaaagccgttcatgaagtagatccaaaaactggtcttttactaaaagatggaaaaggaaaacctaaagctttaaaagaacttcgtacatatgttgacaacatatcaatgctaagcaaggaacaatctaaaggttatgcagatcctaatcttgtttataaattaagagattcaaaaacatttgcttttggtatagaagatttatttgaacttaataaataa

SEQ ID NO: 4 - DNA, 1890 bp, Artificial
  recombinant sequence
ctggttccgcgtggatccaataatgagaccaatgagcctataaaaattgagaatacagcaaataatgaattgattgcaaaagtcaattcattatttaaattgctaaattctagtagtcaaaaaatcaatgatgacagcgcagaagcaaaaacacttaagaataaaatagaaactaaaatcaatgacttaaagtcatttgatcttagtactctaagcgccgatcaattaaacaattatattaatgcattacaattattaataaatcaagcaaataactttattaataagaataattttgaaaatattaaccacaataaaatggaaaatgaaagcgtcactcctaaaatagacaaattacctcctaagcctataaataagccttctattccatttgatccttcgaatataccaacttttcctaattttccttttaatcgctttaataaccaagacaagcacaaatatcctgaacatcatacaaaatttcaaactgttgattctaaaactttgtataaagagttatatgacagaactttttctcttaaatatctaactaaactttcagatggaggactcttaagcaatggttcaggaactacatggttgcttgattatcataaatacacgaatgaaaccaacaaatataaactatttttcgctactaatttacacgttttagctaaatttagtaattctttggaaaaatcagttcaagagaaacttaactattatgacccaactaacgacaaagttgttgctgttgctttaggtaaaagcgctaatgtaactaattttgaccaaaaagccaataaaacaacgtcaaatggttattctcctgttacttattttactaatagtagtgaatttattaattatgaaaaatcaaatgttataactcctatgaatctaacaaatgcaatatcagaaccgaaattggtttttgctgcagttgattttatgaatgatgaagctatcaaaaatattcaaagtggcttgaatgaatcggcagaacagtataaaaactataaaactaaaagtggtgaaattgaacactataaagctgcctatgatgattttgatagtaagaaaaaagttccaattacagttgactttgcagtttttgaagttgatgttgatttagaaaaggctgatgaaacttttaaaagttggattaatgatgctattaaaggtttagatggctacataaatagactaaatgaaacaaactatctcccaaatcaagataaaaatatttctaaatatatgcaaactactgactatgtttcagcttcatatgataggagtaatcaaaacaatttatggaatgcaaaagacatttatattgctggttaccctagtcaaaacaataatgctacttggatgcaaaataatcctaccgaaagaaattctgattcaataacttcttacaaagctggtttaaaaaataaagatacttttgcatttgcaactggaagtattgaagagaaaattggcattccatcaaatgcaacaattaatgataattattggaacagagtaatggcaagttggtatggttatcaatataatattaacttttcatccctatattatggggcttcaggttcattagcctataatgaatttggccaaatgataggtatttataatgctgttacagctagagttgattatggagacttgcttcagtcaggcggaattgctccattcttacaatctagtgatattaaagcaattgaaaacacaatttatgcttacaatcttattgatggaagcaataaatcaatttaccagaatcaaaaaaattcatttagagaaaatcttaaaacactttatcctagtggcttttctgatggttctaaacaaacaaaattattcgatagcatttaa

SEQ ID NO: 5 - DNA, 972 bp, Artificial
  recombinant sequence
ctggttccgcgtggatccccggaattccctattgcattatttgcaccagtattatcagcttcatgtcgtgaaaagcaagattttaaccctaaagattttaaagatgttacattgtctttagcttcagatggtaaaactgattttgctaaaaaatctccatcagagtttgtaaaagaaaataaatcatcatatgcaaaagtgttagaagttacccaaaatggcagtaaaacagacaaatacggttatgagctagttagcgttagacctaatgacaactatggatatttatatgtcgaatataaattatttgacaaaaataacaaaaaaaatataactcctaaaaaatttatttatttacaagactttaagtctaatgtcaaagaacaattatcttctatcaattcaaatttatatttctctaaaactaggaacctaggaaccccagttgataaaaaaacattattagtaaccgaaataataaatgacaaacttcttttagatttgcaaagtaataacctactatcacaagctggagtcaaaatctctaaggatccaaaattagactttggcaatgataatgaaggtatcaaaaaaggtttcgtcaatttatcatttacttacactgttgagcataacgcaagggaaactggcaatgactttccaataaaactagccaaaaagattgaaaattttaaaattgatggcttcactggatatgaaagttttgatcctgataagttcattgaaaaatcatcagccactgatgtaacacttgagaaagttcaaaaagatccaatgtcctatatttctttaaagaatgctggagatacaattttaactgatcttagcgttactaatggtggcaacaaccaagctacaattaaattcaaactacaaaccagtatatttgattctgaaggcaaagaaactaaagtaacttttgataatgaatatgaatataaatacacctga

SEQ ID NO: 6 - DNA, 1107 bp, Artificial
  recombinant sequence
ctggttccgcgtggatccttagcttcttgggaaataacaactagcaagataaataacaaaaatgaaaatgatatttcaatacctaaaaaagaaccactatacaaaaatcattacattaatgaaatgttaaacttttacacaaataacaatgataaccacaaaaaaaattacatttcactccaagaaaataaatcaaatactctttttgatgaattgaaattttcattaacgtactatcctatttttatttctaatagagcattaaataacagacatcaatatcaaacaataattaataaggcaaaagatgctatacaaaagcacttatcagccgattggtattggacaattaataatcttagccacttctggtttattttttcaccatatggtgatttgtacaaaaaaattgataaggaaaaagaattatatgagcaaattcataatgaattaggttcatggattgctcgaatacaaaacaataatcccgaaaagttattaaaatttaaatttccagaagctaaagaaattattagcaagtgggtcgataaaaataagaacgatttcaaaagttatgatccagaaattttaagcaaaaagtctattagcaatatagaatcgtggtatttaatttttgattcaaataaagccataaaaatcttgaaatatattgaaaatgacaagcccaaattgcaaatattaactgacttactaattttcaaaaattctgaaaatattgaggaacaaattacaaagattgaaaatgagataagtagtaagcgcgaatctgatttgacaaaagctattgaaaaaaagaatgaagaggaaattgatgacaaaaaagaagaaaatgatttagggaaaaatttgacatcaagaaatgtgaatagtaatgctgacacagaaacaattgcaaaattaaaaaaggaaattgatctttctaagattttggaaagcgtgattaagctaaaaggcgataaagaattctttgaatttcatgcaacacatcagtataatcaacattttgttgatgcaattgatactataaacaagaatgaactgtcagtatatagatttactataaggaacattgacaatgaaaattag

SEQ ID NO: 7 - DNA, 1425 bp, Artificial
  recombinant sequence
ctggttccgcgtggatcctcatcatggaataatgaagatgacattttcaatttaaaagcaaatacggaagtaaaagccagtgacattttttataaaacctttttatcgcaattaaaagcatatacattagagtctttactcaataacttgcaaagtagtatattgacgcttaatcttcctaataaagttgatgaatttaagttaagtaataataaagatgacattatttttaagtataaaggtaaaagttactcattaaaaaatgttgcaaacaaaataaatggctttgattttaatgaaattttaagaccttttacatatgaaaaagaagatggtaaatttattgtaaaaagagcaaaaaatatcaatgacaagactgatatagatattttatttaaattaaaaacggatgaaaaattaaactattccaacttttttgaatacaaaagcataattttccaaaattactataaaaaaggcttactagatgatctaagtattcctgatttacaatatatgttgcaaagtgcttttgttaatagtagtacacaattccctatgcaagtaacatcaaataatacaagaagcaaggcattttttaagtctaaattccaacaagaaattttagagaaacgactttcaaatgagctaaaaatttataattttgcttctaatggcataattttcgatcatgtcaaatttaataatttaaaaattgataatgatactattaaactcaatattgacttacttgattctaacaataattcccttcttagtgataagtacaaaaatttagaatttaaattaactaacttttccaaaggtcaaagtgacgtctattttgatttaaagactaaggcgaaacttactattgataatgatgaagttaagttcaatgaattagttaataatcctgaaattaaatttaagccaaatccattaacatacagaaccattgatgatttaatgcaccctactaagacatatgaaatatttaatttaaataatactgcaatgctacttagtgaattaaaagatgatattttaatatctaatactccagccaaatttgactttagaatagacaaatttgaaaaaactaaattattaaataactcattatcaattggtaaattagtcataaatgaatctaaaacaaaacaaaagtataattggtattcaattgattttacaccacataaacacattttttcaaatggtttatatttaaaaaatgaattagggacaataaataaaaataaagattcatactttagttattcagtaaatgataatagatttgataataaaggtaatttaagtgttccacaaggaattaaagcaactgattttattgagaattcatttaatgacattgcaaactttttaatttatcaaaataaagataacttattattataa

SEQ ID NO: 8 - DNA, 1767 bp, Artificial
  recombinant sequence
ctggttccgcgtggatccgcttgggataataaggataaagctcacaaaaatcctaataatcaagatcaatctgttagtgaaattcaaaagctaaaagatgagcttcaagatgaaataactaaagcaactagcaagcgtaatcattatcttggcgactatacagactatgcctttaatcaatctgctaaggtaattcaagaagctgtttatgttcatcataatgcaaaaacagttgaagaagttaagaaagctattgaaaaattaaaagcgggaatcaaagaaattgacgcttactttgtactttcaaaagaagaaaaacaaaaacttgccataaaagattttaataaatcactatcagctgccaaggaagctaaagcaaaattaactaagccaagtgcaattgaagtattaaatgattcaattgctaaagctgaatcaaataaagaaggagacccacactattttacaaacctgctaaaatggacttataaatctaaaaaagatattgaagaagcgaaaaaattagaagaaaaaagtgataaggaacttttaaaattatatttatctaaagaatcaaatatacttaatacctttaacagagaatacaatgctagtggttcttttgacgagcaagcagaaaaactaaaagagattttcaaaaaagctaatgaaaacaaaaaagaagattatagagatcaagaagttaagctaagaaaagaaatttctgagattttagagaaaatatcaaaagaagctattactccttctaaagctaatgaattagctaaattagttaaaaatggtgaattaattattggtgacgattataagtacatattatcagatgcatttgctgacaataagaaaatcaaaaaagttactcttggtaaaaaaattgaaaaaatagaaaggggggcttttgataactctggagttgagactataacttttaatgaaaatttaaaatcattagaagggtttaacaataaaaaagttaaagaattaacattgcctaaaaaactagagaaatttgctgcctctaatagcactaaaattactaagttacttcttcctaagacattaaaatcatttgcactaagcagcacatttttagaagaattagaatttgaaccagactttaagtttgacaaagtaagccctgattcatattatgaatcaattgctattaaccagcaattattgccttcgcttaataaaatatatgttagcgatggctctgctaagaaaaatctgctagaaaaattaaataaaaatattacagaatcaaaaaaaataaaactaggagaagattacattaagttcctagcatatattgttaatgtaaaaattgttaaaaagtttgcttcaataactgaagataaaaagaaaaaaattattgaagcactacttgagtcgtcaattaaaaaagaagaaactaaaaaaactaagtctggaattgaaaaaattgttgctgatctaaagagtgaaaatttaaaggacacattagaaaaagttcttgattcatataagtatgaaaagatcagtgctaaaaataaatttgaagtgtttgatgccgctatagaaatgttaaaagacatattgagagcatatggtttttatttaaaaactgaaactattaaaaacgatcctgacctaaaacagtatcatcatgaaacaggtgttactccaggagaaattattaattattttgactcagagaaggaaaaatatgaaaatggtaaaaatggaagtgattcaaaccaatga

SEQ ID NO: 9 - DNA, 2148 bp, Artificial
  recombinant sequence
ctggttccgcgtggatccccggaattcccggtcgacaaaacggctattgacctgattaaaagcctggcgacgacgcagattgtcctgaaccacgaaggcaccatcggccagttcgtggccaacatcttcaactacgcattcgatgctaaagacgcgggttccatgatttacgatcagctgatcaaatcattcggcaccgacaaactgacgcaattcatcagtaaagatgaattttccaccctgttcaaatcaatcttcgtgaaaaaccataaagttattgcgtctatcatgcgtaatgttaccctgacgatgaaacaggacttctacaaataccacaacgctaaatcgatcgcggatgtcctgaaaatctatctggatagccaagacaaaatctacctgatcagtaaagatttctccgtgctgctggaaagctttctgaaagaagaaaccgttaaaaacctgattcagaaactgctgtcaacgcaactgtcgcattataaaatcgatgccaactctgaacagaacaaagcatttttcaaagatctgctggacgaactgccgagtctgattcatgatctgcacattatcccgaacgtgctgaatggcatgtcgaacggcctgaataaacataaaagcctgaccgacctgtttgctgacattctgcagctgacggcgaaatccatcaacttcgaagatttcatcttcgtgcagcgtatcctgaatagctctaccctggaaaaacactcacaagttctgggtgaaaacattaagaaaattgccgaaggcgcaaccacggatgacaccctggttaacaaattcatcgatgacttcggtctgtctaaactgctgacgagcaaaggcatctctgatcatgacgcgaaagatttctttaaactgatgctgaaaagtggtaataccaaaggcgccctggacctgtttatcggtgaactgtttaaaaacgtttccaaactgaaagatgtcaattcatggccgaaactgatttcgacgctgttcaacagcgtgaatgagaaaaaagccaaagaacacatcaaaaaatggatcgttgatgtggttaacgcccgcgactccaaactgtttaatgtcatggcaaaaattaccatcgactcactgaacaaagaaggttatacgattccggtggatgaagaaagtgaaaaactgtccaaattcctggaatcaatctcgaaagcgatcgttggccagaacagcaatgaaaccggcgtcaacggtccgattctggaaatcttcaacgatgttatcgactctatcttcaaaaccctgaaagaactgaaagatagtgatgacgtgctgacgaaaattgccgaagccgttaaaaacggtgccctgaaatttatctctgatggcgacaccatcagtatcgcaaaagttttcgataacatcccgaaattcgaaaaactgctggaaaaagtcgatccgaaatcgtatgctgacttcatcaacgtgatgtttaaatacgcgccgagcgatgaaaacaaaggtctgttcaacgctatcttcaacagcgataaatctaaaggcaataccagcctgggtgcacgtggtttctggggcgtcattcgcggtaaagtgcagcaactgatctcagcatttgtctcgccgctgatgaaaaactacatcgatgaactgctgaaaagcgaaaaagtgtacaaaaccgcgcaggaagttaaagaaaatgtcgaaggctatcaagctgtttggcgcgtgtatgccagcctggccgcaatgctgtatagcaacacgccgtctggtctgttctggaacgccaccaatctgacgtccgaagcatttctgcgcgaaggcttccgtcgcgcctttcaggaagcaatcaaaaacaaagatctgaccaaatacaaagacaattacgcagctattggtctgatcaacgctaaagatgtcaatgacaaattttgggcgggtgtgtctggcctgcgccatagttccattagttattggctgcgtgatcactattacgcgcgcgactacgtgctgatctacatctactacaaagatagtaaagacaccaaatacaacggccagaaaacgaaaaaacaagtgctgattgaagacctgaaaaaaggctttatgccggtggacccgcaaccgaaatga

SEQ ID NO: 10 - protein, 613 aa, Artificial
  recombinant sequence
MVPRGSIKDVKSDNKTPLDLRASSPNIIKESEKVKYVALGDSISAGFDSQLDQDYPGNYDKEKNEVTGISFPTYLASFIKNSNADNSKLESYTNYASSGTTLEDWKFLLTVDDSKLKQLAENDKNKLAYYKSKFGKDIIKKAKEVREELQKANLVTITLAANDFMELVAKKLKNPEFIKQIQNIKANPTNAIAGVSSAFQDTFKEMKKRIDDFSAELKKHVKTENVNFIGYPLPLPHLFVLLDGYLFKNNGSSLIVSQMAVDALNKNIQSQVTKNKFNFINPYDFSFWTDNSNINKLSPTLFDIHPGSFGYKKMAQDVFIKLIVPERNSDKLNENGIKWDNKYIFTDKDSFTTQLEFANKYETVKKVLTDDINGNLFKEDEIFKKFSDKRKNKWEWYKKRVLNSSLVKNILNGAIDSLIQFPTIKEADLSSRLSNFFTKNNNENLNKLKKWLEESEFVPKFLLRSELEFQETYWNKDKKSSSERAKLFDLINIFKNNLLNEANLISLIASFFSSDLIQKEKEELKEIIAEFAQNLIIKNVDEKMLSDAINKAIGNNSSKAFKTENLAKLIKSVLSSKSIKKTIGELFINLANNSKIYGQAKSVVDSSGRIVTD

SEQ ID NO: 11 - protein, 369 aa, Artificial
  recombinant sequence
MVPRGSENGTIKAKEVAEVHLPDNFDIKKLNSSEKRVFKKANSVNLNSVDYTKGTTFEIPGEKDYFLKIYSLTPPSIDENTVVPLDKDKEVSAEINLEFKIIYKDPVENTVELVDGWEKKLSIKNDKFILHSLNSVYNSFESNKASAFEFKNIDWDKLKAKYFDAEIVKWSDGDTPVVKVTSVDPLNQNVKVGDETKIRISGVDTPEKFVGGIKSSNLEHNYALLSSKFAEEALPKGSKVRVYGDSKDAYGRIVGDVFFGDKYQYLYSVEITRSGLTLPYYDNVTLAFSFVKNPVYWEHYALLRLGEALEYAKTNKKGFFKTFALPYWIQSNIYKMKPNGKWTLLDKHSPTNLYNIEGINLIDDSYKNK

SEQ ID NO: 12 - protein, 723 aa, Artificial
  recombinant sequence
MVPRGSNDPKNNSEVIIYQRNANSDKTTEQFEKYNQVNLLSNIKDYFDKHEHLNLISYKGGGKPETVEYSLMMQNNYMSKYINFDTEAFKAIVKKELKLSDKLLSRLKFSYDYNNVTRDPGNNYDVLFKVKVGLPLESNDKSKYESGLYSQQIIDFRIKNVKVKDNDKPFADALKPYDQKIQALTSQDFEVKLENIDDELKQAITKYGIHQLSSKQYEKLLKFSSAKLDEILKDNDKELEIPVGSKKEKYTLKFNKFIEDVVLNDGSLSNAKAKIRIAASLFDSKKKSTVWESGKTVYGLFTVPKEQKLISDLQLAELIDVHTIGNIEHNSDLSTLTKGNLFINVKDANIEKVEIDKITTQDFRNATVTLNVKLKNSQELVKVEKHLGVGRYSLLFDKQFTKQNIKAPAFATEGITTKNLPSIDKTFFGHYNSQLFSGGYASARAFYADNVVEPKYVHIGEDYIAPDFQPVLAPYDGQIVAAYELTTKVVATGVGTVVVIKIPVANLDWSPKEKEIYLNGNNEHIYMSFLHLDAGKTLNNTSLGWQSETVTLGKRTIKVVPTVSADTPTEVKKGQIIGFLGTQDTNGGWMAHAHVNLYTNRNFWLSPNHFNKESKQSNIDKRVKEYKKTDKNKKITYRIVGNAGVEVFLNPKAVHEVDPKTGLLLKDGKGKPKALKELRTYVDNISMLSKEQSKGYADPNLVYKLRDSKTFAFGIEDLFELNK

SEQ ID NO: 13 - protein, 628 aa, Artificial
  recombinant sequence
MVPRGSNNETNEPIKIENTANNELIAKVNSLFKLLNSSSQKINDDSAEAKTLKNKIETKINDLKSFDLSTLSADQLNNYINALQLLINQANNFINKNNFENINHNKMENESVTPKIDKLPPKPINKPSIPFDPSNIPTFPNFPFNRFNNQDKHKYPEHHTKFQTVDSKTLYKELYDRTFSLKYLTKLSDGGLLSNGSGTTWLLDYHKYTNETNKYKLFFATNLHVLAKFSNSLEKSVQEKLNYYDPTNDKVVAVALGKSANVTNFDQKANKTTSNGYSPVTYFTNSSEFINYEKSNVITPMNLTNAISEPKLVFAAVDFMNDEAIKNIQSGLNESAEQYKNYKTKSGEIEHYKAAYDDFDSKKKVPITVDFAVFEVDVDLEKADETFKSWINDAIKGLDGYINRLNETNYLPNQDKNISKYMQTTDYVSASYDRSNQNNLWNAKDIYIAGYPSQNNNATWMQNNPTERNSDSITSYKAGLKNKDTFAFATGSIEEKIGIPSNATINDNYWNRVMASWYGYQYNINFSSLYYGASGSLAYNEFGQMIGIYNAVTARVDYGDLLQSGGIAPFLQSSDIKAIENTIYAYNLIDGSNKSIYQNQKNSFRENLKTLYPSGFSDGSKQTKLFDS

SEQ ID NO: 14 - protein, 323 aa, Artificial
  recombinant sequence
MVPRGSPEFPIALFAPVLSASCREKQDFNPKDFKDVTLSLASDGKTDFAKKSPSEFVKENKSSYAKVLEVTQNGSKTDKYGYELVSVRPNDNYGYLYVEYKLFDKNNKKNITPKKFIYLQDFKSNVKEQLSSINSNLYFSKTRNLGTPVDKKTLLVTEIINDKLLLDLQSNNLLSQAGVKISKDPKLDFGNDNEGIKKGFVNLSFTYTVEHNARETGNDFPIKLAKKIENFKIDGFTGYESFDPDKFIEKSSATDVTLEKVQKDPMSYISLKNAGDTILTDLSVTNGGNNQATIKFKLQTSIFDSEGKETKVTFDNEYEYKYT

SEQ ID NO: 15 - protein, 367 aa, Artificial
  recombinant sequence
MVPRGSLASWEITTSKINNKNENDISIPKKEPLYKNHYINEMLNFYTNNNDNHKKNYISLQENKSNTLFDELKFSLTYYPIFISNRALNNRHQYQTIINKAKDAIQKHLSADWYWTINNLSHFWFIFSPYGDLYKKIDKEKELYEQIHNELGSWIARIQNNNPEKLLKFKFPEAKEIISKWVDKNKNDFKSYDPEILSKKSISNIESWYLIFDSNKAIKILKYIENDKPKLQILTDLLIFKNSENIEEQITKIENEISSKRESDLTKAIEKKNEEEIDDKKEENDLGKNLTSRNVNSNADETIAKLKKEIDLSKILESVIKLKGDKEFFEFHATHQYNQHFVDAIDTINKNELSVYRFTIRNIDNEN

SEQ ID NO: 16 - protein, 474 aa, Artificial
  recombinant sequence
MVPRGSSSWNNEDDIFNLKANTEVKASDIFYKTFLSQLKAYTLESLLNNLQSSILTLNLPNKVDEFKLSNNKDDIIFKYKGKSYSLKNVANKINGFDFNEILRPFTYEKEDGKFIVKRAKNINDKTDIDILFKLKTDEKLNYSNFFEYKSIIFQNYYKKGLLDDLSIPDLQYMLQSAFVNSSTQFPMQVTSNNTRSKAFFKSKFQQEILEKRLSNELKIYNFASNGIIFDHVKFNNLKIDNDTIKLNIDLLDSNNNSLLSDKYKNLEFKLTNFSKGQSDVYFDLKTKAKLTIDNDEVKFNELVNNPEIKFKPNPLTYRTIDDLMHPTKTYEIFNLNNTAMLLSELKDDILISNTPAKFDFRIDKFEKTKLLNNSLSIGKLVINESKTKQKYNWYSIDFTPHKHIFSNGLYLKNELGTINKNKDSYFSYSVNDNRFDNKGNLSVPQGIKATDFIENSFNDIANFLIYQNKDNLLL

SEQ ID NO: 17 - protein, 587 aa, Artificial
  recombinant sequence
MVPRGSAWDNKDKAHKNPNNQDQSVSEIQKLKDELQDEITKATSKRNHYLGDYTDYAFNQSAKVIQEAVYVHHNAKTVEEVKKAIEKLKAGIKEIDAYFVLSKEEKQKLAIKDFNKSLSAAKEAKAKLTKPSAIEVLNDSIAKAESNKEGDPHYFTNLLKWTYKSKKDIEEAKKLEEKSDKELLKLYLSKESNILNTFNREYNASGSFDEQAEKLKEIFKKANENKKEDYRDQEVKLRKEISEILEKISKEAITPSKANELAKLVKNGELIIGDDYKYILSDAFADNKKIKKVTLGKKIEKIERGAFDNSGVETITFNENLKSLEGFNNKKVKELTLPKKLEKFAASNSTKITKLLLPKTLKSFALSSTFLEELEFEPDFKFDKVSPDSYYESIAINQQLLPSLNKIYVSDGSAKKNLLEKLNKNITESKKIKLGEDYIKFLAYIVNVKIVKKFASITEDKKKKIIEALLESSIKKEETKKTKSGIEKIVADLKSENLKDTLEKVLDSYKYEKISAKNKFEVFDAAIEMLKDILRAYGFYLKTETIKNDPDLKQYHHETGVTPGEIINYFDSEKEKYENGKNGSDSN

SEQ ID NO: 18 - protein, 715 aa, Artificial
  recombinant sequence
MVPRGSPEFPVDKTAIDLIKSLATTQIVLNHEGTIGQFVANIFNYAFDAKDAGSMIYDQLIKSFGTDKLTQFISKDEFSTLFKSIFVKNHKVIASIMRNVTLTMKQDFYKYHNAKSIADVLKIYLDSQDKIYLISKDFSVLLESFLKEETVKNLIQKLLSTQLSHYKIDANSEQNKAFFKDLLDELPSLIHDLHIIPNVLNGMSNGLNKHKSLTDLFADILQLTAKSINFEDFIFVQRILNSSTLEKHSQVLGENIKKIAEGATTDDTLVNKFIDDFGLSKLLTSKGISDHDAKDFFKLMLKSGNTKGALDLFIGELFKNVSKLKDVNSWPKLISTLFNSVNEKKAKEHIKKWIVDVVNARDSKLFNVMAKITIDSLNKEGYTIPVDEESEKLSKFLESISKAIVGQNSNETGVNGPILEIFNDVIDSIFKTLKELKDSDDVLTKIAEAVKNGALKFISDGDTISIAKVFDNIPKFEKLLEKVDPKSYADFINVMFKYAPSDENKGLFNAIFNSDKSKGNTSLGARGFWGVIRGKVQQLISAFVSPLMKNYIDELLKSEKVYKTAQEVKENVEGYQAVWRVYASLAAMLYSNTPSGLFWNATNLTSEAFLREGFRRAFQEAIKNKDLTKYKDNYAAIGLINAKDVNDKFWAGVSGLRHSSISYWLRDHYYARDYVLIYIYYKDSKDTKYNGQKTKKQVLIEDLKKGFMPVDPQPK

SEQ ID NO: 19 - DNA, 1791 bp, Artificial
  recombinant sequence
attaaagatgtgaaaagtgacaataaaactcctttagatttaagagcgtcaagccccaacattattaaagagtcagaaaaagttaaatatgttgcattaggtgattctatatcagccggttttgactcacaattagatcaagattacccaggtaattatgataaagaaaaaaacgaagtaactggtatttcattccctacctatttagcttcatttattaaaaattcaaatgccgataattctaaattagaatcatatacaaactatgcatcttcaggcacgaccttagaagattgaaaatttttacttactgttgatgattctaaattaaagcaattagcagaaaatgataagaataagctagcttattataagtctaaatttggcaaagacataattaaaaaggccaaagaagtaagagaagaattgcaaaaggctaatttggtaactataactttggctgccaatgactttatggaattagttgctaaaaaactaaaaaatcctgaatttataaagcagatacaaaatatcaaagccaatccaactaatgcaatagctggagttagttcagcatttcaagacacattcaaagaaatgaagaaaagaatagatgacttttctgctgagttaaaaaaacatgtaaagactgaaaatgtcaattttataggctacccactgccacttccacatttatttgttttattagatggctatttatttaagaataatggatcatcattaattgttagtcaaatggctgttgatgcactaaataaaaatattcagtcacaagttactaaaaataaatttaattttattaatccatatgatttttcattctgaacagataattctaatataaacaaattgtctcctacattatttgatattcaccctggcagttttggctacaaaaaaatggcacaggatgtgtttattaagcttattgttcctgaaagaaacagcgataaattaaatgagaatggcataaagtgagataacaagtacatttttactgataaagattcatttacaactcaattagagtttgctaacaaatatgagacagttaaaaaagttttaaccgatgatattaatggaaatctctttaaagaagacgaaatattcaaaaagtttagcgataaaagaaaaaataaatgagaatgatacaagaaaagagttttaaacagctcattagttaaaaacatattaaatggcgctattgattcattaattcaatttcccacaataaaggaagccgatctcagtagcagattaagtaatttttttacgaaaaacaataatgaaaatctaaacaaattaaaaaaatggttagaggaatcagaatttgttcctaaattcttgcttaaatctgagcttgagtttcaagaaacttattgaaataaagacaaaaagtcaagtagcgagagagccaaacttttcgatttaataaacattttcaagaataatctactaaatgaagctaatttaatttcattaattgcttcattctttagttctgacttaattcaaaaagaaaaagaagaattaaaagaaataatagcggaatttgcacagaatttaattattaaaaatgttgatgaaaaaatgttgtctgatgctataaataaagcaattgggaataactcatcaaaggcttttaaaactgaaaatcttgctaaattgattaaatcagttttaagcagtaagagcattaaaaaaacaatcggagaattattcattaaccttgcaaataattctaagatttatggacaagctaaatcagtt

SEQ ID NO: 20 - DNA, 2112 bp, Mycoplasma bovis
aaaactgcaattgatttaattaaatcacttgcaacaactcaaattgttcttaatcatgaaggtacaatcggccaatttgtagcgaacatatttaattatgcttttgatgcaaaagatgctggttcaatgatatatgatcagctaattaagtcatttggcactgacaaattaactcaatttatttcaaaagatgagttcagtactttattcaagtcaatttttgtcaaaaatcacaaagttattgcttctataatgcgaaatgtaacattaacaatgaagcaagatttttataaatatcacaatgctaaatcaattgctgatgttcttaaaatatatttagatagccaggataaaatttatctaatttctaaagacttttcagtcttgcttgaaagctttttgaaagaagaaactgtcaaaaatcttattcaaaaattactcagtacacaactaagccactataaaattgatgcaaatagtgagcaaaacaaagcattcttcaaggatttattggatgaattgccttcattaattcatgacttgcacattattccaaatgtgctaaatggtatgagtaacggattaaataaacataaatcattgactgatttatttgctgatatattacaacttacagccaaatctataaactttgaagactttatttttgtacagagaattttaaactcttcaacattagaaaagcattcacaagtcttaggtgagaatataaagaaaattgctgagggcgcaactacagatgataccttagtaaacaaatttattgatgattttggtctttctaagttattaacatcaaagggaatctctgatcatgatgctaaagatttctttaaattaatgcttaaatctggtaacacaaaaggcgcattagacttatttataggtgaattattcaaaaatgtaagtaaattaaaagatgttaattcatggccaaaactaatttcaacattatttaattctgttaatgagaaaaaagccaaagaacacattaagaagtgaattgtagatgttgttaacgcaagagacagcaaactatttaatgtgatggcaaaaataacaatagattcgctaaataaagaaggctatacaattcctgttgatgaagaatcagagaaattaagtaagttcttagagtctatttcaaaggcaatagttggccaaaacagcaatgaaacaggcgtaaacggtccaattcttgaaatatttaatgatgttatagatagcatatttaaaactcttaaagaattaaaagattctgatgatgtccttacaaaaattgcagaagcagttaaaaatggtgccttaaagttcataagcgatggagatactatttcaattgctaaggtatttgataacattcctaagtttgaaaaacttttagaaaaagtagatcctaaatcatatgctgactttatcaatgttatgtttaaatatgcaccttcagatgaaaataaaggcctatttaatgcaatattcaactcagataaatcgaaaggcaatacatctttaggggctcgtggcttctgaggagttataagaggtaaggttcaacagttaatttctgcatttgtttcgccattaatgaagaattatattgatgagctattaaaatctgaaaaggtctataaaacagcacaagaagtaaaagaaaatgttgaaggatatcaagcagtttgaagagtctatgcttcattagctgcaatgttatattcaaacacacctagtggcttgttctggaatgcaactaatttaacttctgaagcatttttaagggaaggttttagaagagcatttcaagaggcaataaaaaataaggacctaactaaatataaagataactatgcagctataggactaattaatgctaaagatgtaaatgacaaattttgagcaggtgtttctggtctacgtcatagttcaattagttattgactaagagaccattattatgcacgtgattatgtgttgatttatatctattacaaagactctaaagacacaaaatataacggacaaaaaaccaaaaagcaagttcttattgaagatctaaaaaaaggttttatgccagttgacccacaacctaaataa

(19) United States
(12) Patent Application Publication
(10) Pub. No.: US 2024/0183852 A1
(43) Pub. Date: Jun. 6, 2024

(54) MULTIPLEX IMMUNOASSAY FOR THE DETECTION OF MYCOPLASMA BOVIS INFECTION

(71) Applicants: Pictor Limited, Auckland (NZ); The University of Melbourne, Melbourne (AU)

(72) Inventors: Anna Kanci Condello, Melbourne (AU); Glenn Francis Browning, Melbourne (AU); James Yazah Adamu, Melbourne (AU); Jimena Maria Tejerina, Melbourne (AU); Kelly Anne Tivendale, Melbourne (AU); Wasala Mudiyanselage Nadeeka Jumari Wawegama, Melbourne (AU); Philip Francis Markham, Melbourne (AU); Sandeep Kumar Vashist, Aachen (DE)

(21) Appl. No.: 18/285,490

(22) PCT Filed: Apr. 8, 2022
(86) PCT No.: PCT/NZ22/50038

§ 371 Date: Oct. 3, 2023

(30) Foreign Application Priority Data
Apr. 9, 2021 (NZ) 774831

(51) Int. Cl. G01N 33/569; C07K 16/12; G01N 33/543; G01N 33/58; G01N 33/68

(52) U.S. Cl. CPC G01N 33/56933; C07K 16/1253; G01N 33/54326; G01N 33/581; G01N 33/6854; G01N 2469/20

(57) ABSTRACT

Mycoplasma bovis infection in cattle is a major production limiting disease of cows and calves. M. bovis can cause a range of conditions in cattle. including mastitis in dairy cows. arthritis in cows and calves. pneumonia in calves, and various other diseases including late-term abortion. The present invention provides methods for detecting multiple anti-M. bovis antibodies in a single multiplex immunoassay.

### FIELD OF THE INVENTION

The present invention relates generally to multiplex immunoassays and more specifically to a multiplex immunoassay (MIA) for the detection of a Mycoplasma bovis infection.

### BACKGROUND INFORMATION

Mycoplasma bovis is one of the smallest free-living organisms found in nature. M. bovis does not have a cell wall and is therefore resistant to penicillin and other beta lactam antibiotics. M. bovis mainly affects cattle and has little effect on other production animals, but other animals can be carriers. M. bovis can cause a range of conditions in cattle, including mastitis in dairy cows, arthritis in cows and calves, pneumonia in calves, and various other diseases including late-term abortion. Infection is usually introduced to herds by M. bovis infected cattle that appear to be clinically healthy but are shedding M. bovis into the environment or transmitting it via contaminated milk.

The current serological diagnosis of M. bovis infection is done via the detection of antibodies against a single protein antigen. Therefore, infected animals with a poor or delayed antibody response against this single antigen may not be detected. Moreover, as the immune responses differ between individuals, antibodies against different antigens of the pathogen are generated. Therefore, the current on-market singleplex immunoassays have limited sensitivity as they rely on the fact that all infected cattle would respond equally well to the same antigen of the pathogen.

Currently, the best available test is the IDvet ID Screen® Mycoplasma bovis Indirect ELISA, which employs the MilAab recombinant M. bovis antigen. However, this test has only a modest sensitivity on serum samples, and a lower sensitivity for bulk milk testing.

There is a need for a M. bovis multiplex immunoassay that will allow the detection of antibodies against the various antigens of the pathogen. Such an assay would lead to higher detection sensitivity and improved diagnostic performance, thereby obviating the limitation of existing commercial immunoassays.

### SUMMARY OF THE INVENTION

The present invention is based on the seminal discovery of using a multiplex immunoassay for the detection of Mycoplasma bovis infection in cattle.

In one embodiment, the present invention provides a substrate with at least two capture elements specific for Mycoplasma bovis (M. bovis) on the substrate, each capture element corresponding to and being able to bind a target analyte, the substrate further optionally having a plurality of control elements including at least one fiduciary marker, at least one negative control to monitor background signal, at least one negative control to monitor assay specificity, at least one positive colorimetric control, at least one positive control to monitor assay performance and any combination thereof. In one aspect, the capture elements bind target analytes, wherein the target analytes are indicative of an M. bovis infection. In another aspect, the capture element is a protein, lipoprotein, glycoprotein, a protein fragment, a peptide, a polypeptide, a polypeptide fragment, antigen, an antigen fragment, an antigenic determinant, an epitope, a hapten, an immunogen, an immunogen fragment, or any combination thereof.

In certain aspects, the capture element is MilAab (SEQ ID NO: 10), MBOVGP45_310 (SEQ ID NO: 11), MBOVGP45_320 (SEQ ID NO: 12), MBOVGP45_0402 (SEQ ID NO: 13), MBOVGP45_0117 (SEQ ID NO: 14), MBOVGP45_0353 (SEQ ID NO: 15), MBOVGP45_0416 (SEQ ID NO: 16), MBOVGP45_0565 (SEQ ID NO: 17), and/or MBOVGP45_0710-EF (SEQ ID NO: 18), an epitope thereof or a combination thereof. In specific aspects, the capture element is MilAab (SEQ ID NO:10), MBOVPG45_310 (SEQ ID NO:11), MBOVGP45_320 (SEQ ID NO:12), MBOVGP45_0402 (SEQ ID NO:13), and/or MBOVPG45_0117 (SEQ IP NO: 14). In an additional aspect, the target analyte is an antibody or an antibody fragment. In a further aspect, the substrate is a solid or porous substrate. In certain aspects, the solid substrate is a paramagnetic bead, microtiter plate, microparticle, or magnetic bead.

In an additional embodiment, the present invention provides a kit for detecting a plurality of target analytes in a sample, having a substrate having at least two capture elements specific for Mycoplasma bovis (M. bovis) on the substrate, each capture element corresponding to and being able to bind a target analyte, and optionally one or both of a background reducing reagent, and a colorimetric detection system. In one aspect, the kit includes one or more items including a wash solution, one or more antibodies for detection of antigens, ligands or antibodies bound to the capture elements or for detection of the positive controls, software for analyzing captured target analytes, a protocol for measuring the presence of target analytes in samples, a sample diluent, blotting TMB (3,3′,5,5′-tetramethylbenzidine) and/or a secondary antibody. In some aspects, the antibodies for detection are antibody-binding protein conjugates, antibody-enzyme label conjugates, or any combination thereof. In an additional aspect, the sample is a milk or a blood sample. In a further aspect, the blood sample is serum or plasma. In one aspect, the substrate is a solid or porous substrate. In certain aspects, the solid substrate is a paramagnetic bead, microtiter plate, or microparticle. In an additional aspect, the colorimetric detection system is horse radish peroxidase (HRP)-labelled anti-bovine IgG Ab. In specific aspects, the secondary antibody has at least one anti-bovine IgG antibody. In a further aspect, the kit detects a Mycoplamsa bovis infection in a mammal. In certain aspects, the mammal is bovine.

In a further embodiment, the present invention provides a method for processing a microarray by providing a substrate having at least two capture elements specific for Mycoplasma bovis (M. bovis) on the substrate, each capture element corresponding to and being able to bind a target analyte; adding at least one sample to the substrate; and processing the substrate such that a detectable result is given by two or more of at least one fiduciary marker, at least one positive colorimetric control, and at least one positive control to monitor assay performance. In one aspect, the sample is milk, serum, or plasma. In certain aspects, the capture element is MilAab (SEQ ID NO: 10), MBOVGP45_310 (SEQ ID NO: 11), MBOVGP45_320 (SEQ ID NO: 12), MBOVGP45_0402 (SEQ ID NO: 13), MBOVGP45_0117 (SEQ ID NO: 14), MBOVGP45_0353 (SEQ ID NO: 15), MBOVGP45_0416 (SEQ ID NO: 16), MBOVGP45_0565 (SEQ ID NO: 17), MBOVGP45_0710-EF(SEQ ID NO: 18), an epitope thereof or a combination thereof. In specific aspects, the capture element is MilAab (SEQ ID NO:10), MBOVPG45_310 (SEQ ID NO:11), MBOVGP45_320 (SEQ ID NO:12), MBOVGP45_0402 (SEQ ID NO:13), and/or MBOVPG45_0117 (SEQ IP NO: 14).

In another embodiment, the present invention provides a method for detecting an analyte in a sample by providing a substrate having at least two capture elements specific for Mycoplasma bovis (M. bovis) on the substrate, each capture element corresponding to and being able to bind a target analyte, adding at least one sample to the substrate, and processing the substrate such that a detectable result is provided. In one aspect, the sample is milk, serum, or plasma. In an additional aspect, the detectable result includes two or more of at least one fiduciary marker, at least one positive colorimetric control, and at least one positive control to detect an analyte in the sample. In certain aspects, the capture element is MilAab (SEQ ID NO: 10), MBOVGP45_310 (SEQ ID NO: 11), MBOVGP45_320 (SEQ ID NO: 12), MBOVGP45_0402 (SEQ ID NO: 13), MBOVGP45_0117 (SEQ ID NO: 14), MBOVGP45_0353 (SEQ ID NO: 15), MBOVGP45_0416 (SEQ ID NO: 16), MBOVGP45_0565 (SEQ ID NO: 17), MBOVGP45_0710-EF (SEQ ID NO: 18) or a combination thereof. In specific aspects, the capture element is MilAab (SEQ ID NO:10), MBOVPG45_310 (SEQ ID NO:11), MBOVGP45_320 (SEQ ID NO:12), MBOVGP45_0402 (SEQ ID NO: 13), and/or MBOVPG45_0117 (SEQ IP NO: 14). In a further aspect, detecting the analyte is indicative of a Mycoplasma bovis infection.

In one embodiment, the present invention provides an isolated peptide including MBOVGP45_320 (SEQ ID NO: 12), MBOVGP45_0402 (SEQ ID NO: 13), MBOVPG45_0117 (SEQ IP NO: 14), MBOVGP45_0353 (SEQ ID NO: 15), MBOVGP45_0416 (SEQ ID NO: 16), MBOVGP45_0565 (SEQ ID NO: 17).

In an additional embodiment, the present invention provides an antibody that binds a peptide including MBOVGP45_310 (SEQ ID NO: 11), MBOVGP45_320 (SEQ ID NO: 12), MBOVGP45_0402 (SEQ ID NO: 13), MBOVGP45_0117 (SEQ ID NO: 14), MBOVGP45_0353 (SEQ ID NO: 15), MBOVGP45_0416 (SEQ ID NO: 16), or MBOVGP45_0565 (SEQ ID NO: 17). In an additional aspect, the antibody is monoclonal.

In a further embodiment, the present invention provides a nucleic acid encoding a peptide including MBOVGP45_320 (SEQ ID NO: 12), MBOVGP45_0402 (SEQ ID NO: 13), MBOVPG45_0117 (SEQ IP NO: 14), MBOVGP45_0353 (SEQ ID NO: 15), MBOVGP45_0416 (SEQ ID NO: 16) or MBOVGP45_0565 (SEQ ID NO: 17).

In one embodiment, the present invention provides a method of detecting a Mycoplasma bovis infection in cattle by adding at least one sample to a substrate having at least two capture elements specific for M. bovis on the substrate, each capture element corresponding to and being able to bind a target analyte; and processing the substrate such that a detectable result is given by two or more of at least one fiduciary marker, at least one positive colorimetric control, and at least one positive control to monitor assay performance, thereby detecting a M. bovis infection. In one aspect, the sample is milk, serum, or plasma. In an additional aspect, the capture element is MilAab (SEQ ID NO: 10), MBOVGP45_310 (SEQ ID NO: 11), MBOVGP45_320 (SEQ ID NO: 12), MBOVGP45_0402 (SEQ ID NO: 13), MBOVGP45_0117 (SEQ ID NO: 14), MBOVGP45_0353 (SEQ ID NO: 15), MBOVGP45_0416 (SEQ ID NO: 16), MBOVGP45_0565 (SEQ ID NO: 17), MBOVGP45_0710-EF(SEQ ID NO: 18), an epitope thereof or a combination. In certain aspects, the capture element is MilAab (SEQ ID NO:10), MBOVPG45_310 (SEQ ID NO:11), MBOVGP45_320 (SEQ ID NO:12), MBOVGP45_0402 (SEQ ID NO:13), and/or MBOVPG45_0117 (SEQ IP NO: 14).

### BRIEF DESCRIPTION OF THE DRAWINGS

FIG. 1 shows a schematic of membrane-free MIA for the detection of anti-M. bovis antibodies in a milk, serum or plasma sample from a M. bovis infected cow.

FIGS. 2A-E show the membrane-free MIA procedure. FIG. 2A: Printing of M. bovis recombinant antigens (Ag) on the surface of the well of a 96-well MTP. FIG. 2B: Blocking of the surface after the printing of Ag. FIG. 2C: Detection of anti-M. bovis antibody (Ab) in cow's milk, serum or plasma sample. FIG. 2D: Detection of specifically bound anti-M. bovis Ab by binding with a HRP-labeled detection Ab. FIG. 2E: Generation of colorimetric array spots by the addition of precipitating TMB substrate.

FIG. 3 shows the membrane-free M. bovis MIA. The recombinant M. bovis Ag MilAab, MBOVPG45_320, MBOVPG45_310 and MBOVPG45_0402, are printed as duplicate spots in each well of the MTP of detachable 8-well modules of a 96-well MTP.

### DETAILED DESCRIPTION OF THE INVENTION

The present invention is based on the seminal discovery of using a multiplex immunoassay for detection of Mycoplasma bovis infection in cattle.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods and/or steps of the type described herein, which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. The preferred methods and materials are now described.

The invention describes the multiplex immunoassay (MIA) procedures for the detection of multiple analytes for the diagnosis of M. bovis infection in cattle. It involves the detection of bovine analytes, anti-M. bovis IgG, in milk or serum via a multiplex immunoassay.

The current serological diagnosis of M. bovis infection involves the detection of antibodies against a single protein antigen of M. bovis. However, the assay does not have a high sensitivity as not all the infected animals show a strong immunogenic response against a single protein antigen. Therefore, multiple immunoassays after every few days need to be performed to improve the sensitivity of the immunoassay, which takes more time, efforts, and costs. This low specificity immunoassay is used as a screening test in the first-tier testing. The identified positive samples are then confirmed using a highly specific and sensitive test in the second-tier testing.

The invention MIA, incorporating the use of multiple antigens of M. bovis to detect antibodies produced in animals against M. bovis, is an ideal solution to develop a very high sensitivity immunoassay. The MIA includes the binding of different M. bovis antigens in the same 96-well microtiter plate well, followed by the simultaneous detection of anti-M. bovis antibodies against each of the antigens. The MIA has high sensitivity as it will be able to detect anti-M. bovis antibodies in almost all infected animals. Moreover, simultaneous testing for responses that may develop at different times after initial infection can be performed; for example the longitudinal profiling of antibodies generated against each of the coated antigens can be performed for several weeks. This enables a more precise diagnosis of infected animals based on their immunological profiling and allows the detection of infected animals that have failed to mount an antibody response against the single protein that is being used in the current commercial immunoassay.

In one embodiment, the present invention provides a substrate having at least two capture elements specific for Mycoplasma bovis (M. bovis) on the substrate, each capture element corresponding to and being able to bind a target analyte, the substrate further optionally having a plurality of control elements comprising: at least one fiduciary marker, at least one negative control to monitor background signal, at least one negative control to monitor assay specificity, at least one positive colorimetric control, at least one positive control to monitor assay performance and any combination thereof. In one aspect, the capture elements bind target analytes, wherein the target analytes are indicative of an M. bovis infection. In another aspect, the capture element is a protein, lipoprotein, glycoprotein, a protein fragment, a peptide, a polypeptide, a polypeptide fragment, antigen, an antigen fragment, an antigenic determinant, an epitope, a hapten, an immunogen, an immunogen fragment, or any combination thereof. In certain aspects, the capture element is MilAab (SEQ ID NO: 10), MBOVGP45_310 (SEQ ID NO: 11), MBOVGP45_320 (SEQ ID NO: 12), MBOVGP45_0402 (SEQ ID NO: 13), MBOVGP45_0117 (SEQ ID NO: 14), MBOVGP45_0353 (SEQ ID NO: 15), MBOVGP45_0416 (SEQ ID NO: 16), MBOVGP45_0565 (SEQ ID NO: 17), MBOVGP45_0710-EF (SEQ ID NO: 18), an epitope thereof or a combination thereof. In specific aspects, the capture element is MilAab (SEQ ID NO:10), MBOVPG45_310 (SEQ ID NO:11), MBOVGP45_320 (SEQ ID NO:13), or MBOVGP45_0402 (SEQ ID NO:13). In an additional aspect, the target analyte is an antibody or an antibody fragment. In a further aspect, the substrate is a solid or porous substrate. In certain aspects, the solid substrate is a paramagnetic bead, microtiter plate, microparticle, or magnetic bead.

As used herein, the term “substrate” is any surface that supports an immunoassay. The substrate of the invention may be a solid substrate or a porous substrate, for example.

In certain aspects, the substrate is a solid substrate. Examples of solid substrates include, but are not limited to, 96 well microtiter plates, glass, microbeads, nano/micro- particles and magnetic beads. In one aspect, the bottom of a 96 well microtiter plate is made up of polystyrene, polydimethylsiloxane (PDMS), poly (methyl methacrylate) (PMMA), polycarbonate, cyclic polyolefins, Zeonor, Zeonex, or cellulose acetate. In various aspects, the solid substrate maybe glass beads, nano-/microparticles, magnetic beads or paramagnetic beads.

The assay elements (control and capture elements) are placed on the substrate surface, with or without an adapter molecule between the substrate and the element. Preferably, the assay elements bind to the substrate by covalent or non-covalent interaction. One of skill in the art will recognize the methods of placing assay elements on the substrate include printing, spotting or other techniques known in the art. For purposes of the present application, the term “printing” can be used to include any of the methods for placing the assay elements on a membrane.

The terms “array” or “microarray” as used herein refer to a collection of multiple assay elements on a substrate. Specifically, an array is a collection of capture elements and/or control elements on a substrate.

In various aspects, the elements on the array are placed on the substrate in discrete areas of between 100 μm to 500 μm in diameter. More preferably, the discrete areas are between 350 μm to 400 μm in diameter. In certain aspects, the discrete areas of the array are placed in a 5×5 grid. In one aspect, the array comprises up to nine control elements and two replicates of each of eight different capture elements. In one aspect, the capture elements are printed in two or more replicates of four different capture elements and multiples thereof.

As used herein, the term “assay element” refers to any of a number of different elements for use in an array of the invention. Exemplary assay elements include, but are not limited to, capture elements and control elements.

The term “capture element” refers to a molecule that is able to bind to a target analyte. Examples of useful capture elements include proteins, protein fragments, polypeptides, polypeptide fragments, binding proteins, binding protein fragments, antibodies (polyclonal, monoclonal, or chimeric), antibody fragments, antibody heavy chains, antibody light chains, single chain antibodies, single-domain antibodies (a VHH for example), Fab antibody fragments, Fc antibody fragments, Fv antibody fragments, F(ab′)2 antibody fragments, Fab′ antibody fragments, single-chain Fv (scFv) antibody fragments, antibody binding domains, antigens, antigenic determinants, epitopes, haptens, immunogens, immunogen fragments, and binding domains. Useful capture elements will correspond to and are able to bind a specific target analyte, such as a molecule or class of molecules that are present in a sample to be tested.

In one embodiment, the capture element is a protein, a protein fragment, a binding protein, a binding protein fragment, an antibody, an antibody fragment, an antibody heavy chain, an antibody light chain, a single chain antibody, a single-domain antibody (a VHH for example), a Fab antibody fragment, an Fc antibody fragment, an Fv antibody fragment, a F(ab′)2 antibody fragment, a Fab′ antibody fragment, a single-chain Fv (scFv) antibody fragment, an antibody binding domain, an antigen, an antigenic determinant, an epitope, a hapten, an immunogen, an immunogen fragment, and a binding domain.

In another aspect, the capture elements may comprise antibodies or fragments thereof that are immobilized on the substrate surface and are specific for different antigens or ligands that may be present in a sample. In certain aspects, the capture elements may comprise antigens or ligands and the assay involves the detection of specific antibodies that may be present in a sample. In various aspects, the capture elements may comprise of a receptor or a subunit of a receptor that binds a specific ligand.

Specifically, the capture element can be SEQ ID NO: 10, SEQ ID NO:11, SEQ ID NO:12, SEQ ID NO:13, SEQ ID NO:14, SEQ ID NO:15, SEQ ID NO:16, SEQ ID NO:17, SEQ ID NO: 18, an epitope thereof or any combination thereof.

The terms “sequence identity” or “percent identity” are used interchangeably herein. To determine the percent identity of two polypeptide molecules or two polynucleotide sequences, the sequences are aligned for optimal comparison purposes (e.g., gaps can be introduced in the sequence of a first polypeptide or polynucleotide for optimal alignment with a second polypeptide or polynucleotide sequence). The amino acids or nucleotides at corresponding amino acid or nucleotide positions are then compared. When a position in the first sequence is occupied by the same amino acid or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). In some embodiments the length of a reference sequence (e.g. SEQ ID NOs: 1-20) aligned for comparison purposes is at least 80% of the length of the comparison sequence, and in some embodiments is at least 90% or 100%. In an embodiment, the two sequences are the same length.

Ranges of desired degrees of sequence identity are approximately 80% to 100% and integer values in between. Percent identities between a disclosed sequence and a claimed sequence can be at least 80%, at least 85%, at least 90%, at least 95%, at least 96%, at least 97%, at least 98%, at least 99%, at least 99.5%, or at least 99.9%. In general, an exact match indicates 100% identity over the length of the reference sequence (e.g., SEQ ID NOs: 1-20).

Polypeptides and polynucleotides that are about 85, 86, 87, 88, 89, 90, 91, 92, 93, 94, 95, 96, 97, 98, 99 99.5% or more identical to polypeptides and polynucleotides described herein are embodied within the disclosure.

For example, a polypeptide can have 80%, 85%, 90%, 91%, 92%, 93%, 94%, 95%, 96%, 97%, 98%, 99% or more identity to SEQ ID NO: 10, SEQ ID NO: 11, SEQ ID NO: 12, SEQ ID NO:13, SEQ ID NO:14, SEQ ID NO: 15, SEQ ID NO: 16, SEQ ID NO: 17 or SEQ ID NO: 18. A nucleotide can have 80%, 85%, 90%, 91%, 92%, 93%, 94%, 95%, 96%, 97%, 98%, 99% or more identity to SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 19 or SEQ ID NO: 20.

Variants of the disclosed sequences also include peptides, or full length proteins, that contain substitutions, deletions, or insertions into the protein backbone, that would still leave at least about 70% homology to the original protein over the corresponding portion. A yet greater degree of departure from homology is allowed if like-amino acids, i.e. conservative amino acid substitutions, do not count as a change in the sequence. Examples of conservative substitutions involve amino acids that have the same or similar properties. Illustrative amino acid conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine to leucine.

As used herein, the terms “biomarker” refers to any substance used as an indicator of a biological state. Thus, a biomarker can be any substance whose detection indicates a particular disease state (for example, the presence of an antibody may indicate an infection). Furthermore, a biomarker can be indicative of a change in expression or state of a protein that correlates with the risk or progression of a disease, or with the susceptibility of the disease to a given treatment. Once a proposed biomarker has been validated, it can be used to diagnose disease risk, presence of disease in an individual, or to tailor treatments for the disease in an individual (e.g., choices of drug treatment or administration regimes). In evaluating potential drug therapies, a biomarker may be used as a surrogate for a natural endpoint such as survival or irreversible morbidity. If a treatment alters the biomarker, which has a direct connection to improved health, the biomarker serves as a “surrogate endpoint” for evaluating clinical benefit. In one aspect, the target analyte is a biomarker.

In one embodiment, the target analyte is a protein, a protein fragment, a peptide, a polypeptide, a polypeptide fragment, an antibody, an antibody fragment, an antibody binding domain, an antigen, an antigen fragment, an antigenic determinant, an epitope, a hapten, an immunogen, an immunogen fragment, or any combination of any two or more thereof. In one aspect, the target analyte is an antibody to M. bovis.

Capture elements specific for a target analyte are used to detect the presence or absence of the analyte in a sample. A wide range of complementary binding or coupling partners are known, with the choice of capture elements determined by the analytes to be detected, the requirement for adapter molecules and the level of specificity required for the assay. In various aspects, the capture elements are specific for binding/detecting M. bovis.

The term “control element” refers to an element that is used to provide information on the function of the assay, for example binding specificity, the level of non-specific background binding, the degree of binding cross-reactivity, and the performance of assay reagents and the detection system. Preferred controls useful herein include at least one negative control to monitor background signal, at least one negative control to monitor assay specificity, at least one positive colorimetric control, and at least one positive control to monitor assay performance.

The substrate of the invention comprises at least one fiduciary marker that will always be detectable on the substrate, preferably detectable irrespective of the performance of the assay or processing of the substrate.

The term “fiduciary marker” refers to a colored marker or label that will always be detectable on the substrate, preferably irrespective of the performance of the assay or processing of the substrate. The use of at least one fiduciary marker will obviate the necessity of this element being detected based on successful array processing, in comparison to the positive colorimetric controls. The fiduciary marker is therefore a “true” positive control that would always be detectable regardless of array processing and can be used to orient and help to grid the array.

In preferred aspects, the fiduciary marker is a dye, dye-conjugated protein or a chromogenic protein such as hemoglobin.

The term “negative control” refers to an element comprising print buffer or an unrelated protein to which no complementary binding partner is intended to be present in the assay. Any detectable signal from the negative control can be used to determine the background threshold of the assay and the accuracy of any positive results. In one aspect, the negative control to monitor background signal is print buffer. The print buffer is a solution used to carry and print the capture elements and control elements onto the substrate and may comprise buffered saline, glycerol and a surfactant, preferably a polysorbate surfactant such as Tween 20. The blocking solution is used to reduce non-specific protein binding to the substrate surface and preferably comprises skim milk, casein, bovine serum albumin, gelatins from fish, pigs or other species, dextran or any mixture of any two or more thereof, preferably in a solution of phosphate buffered saline and a surfactant such as Tween 20.

The term “control capture element” refers to a capture element that functions as a control, either a negative control that should not bind any analyte or a positive control that will bind a non-target analyte.

The substrate of the invention also comprises at least one control to monitor assay performance. The control is intended to provide information of the efficiency of the complementary binding interactions or the quality or performance of the reagents used.

The term “control to monitor assay performance” refers to an element that forms one part of a complementary binding interaction during an assay and is intended to provide information on the accuracy of the assay result. In one embodiment, the positive control to monitor assay performance comprises one binding partner of a complementary binding pair, where the other binding partner is a sample component or an assay reagent. The assay performance control is preferably a target analyte, a binding partner corresponding to and able to bind a non-target analyte that will be present in the sample, a binding partner corresponding to and able to bind an assay reagent, and a colorimetric enzyme label, or any combination of any two or more thereof. An example of a binding partner corresponding to and able to bind a non-target analyte that will be present in the sample is an anti-Ig antibody that will bind an immunoglobulin present in a serum sample, therefore confirming a sample has been added. An example of a binding partner corresponding to and able to bind an assay reagent is an anti-Ig antibody that will bind a secondary immunoglobulin that is used to process the assay, such as biotinylated anti-target analyte antibody. Another example of a binding partner corresponding to and able to bind an assay reagent is a biotinylated antibody that will bind a streptavidin-peroxidase conjugate that is used to process the assay.

In one aspect, the assay performance control comprises one binding partner of a complementary binding pair, wherein the other binding partner is an assay reagent. The assay performance control is preferably the target analyte, a non-specific binding partner or a colorimetric enzyme label.

In another aspect, the complementary binding partners comprise antibody-antigen interactions or antibody-ligand interactions.

The substrate of the invention also comprises at least one control to monitor assay specificity. The control is intended to provide information of the specificity of binding between the capture element and the target analyte, or between the binding partners of the assay detection steps.

The term “control to monitor assay specificity” refers to an element that is closely related to at least one binding partner of a complementary binding pair present in the assay and is intended to provide information of the specificity of the complementary binding. This control is a negative control that is not expected to generate a detectable result during normal assay processing. For example, in an antibody array for antigen detection, the assay specificity control would comprise an antibody that should not bind any antigen in the sample. Alternatively, in an antigen array for antibody detection, the assay specificity control would comprise an antigen that should not bind any antibody in the sample.

In one aspect, the assay specificity control is Mycobacterium phlei protein extract. In another aspect, the assay specificity control is Mycobacterium tuberculosis antigen.

The term “positive colorimetric control” as used herein refers to an enzyme or enzyme conjugate that provides a detectable signal upon the enzyme substrate's addition.

In one embodiment, the positive colorimetric control is an enzyme label conjugate capable of reacting with a colorimetric substrate, comprising an enzyme comprising horseradish peroxidase, alkaline phosphatases, β-D-galactosidase or glucose oxidase.

The identity of the assay controls will be dependent on the type of array, the identity of the target analyte, and the type of sample to be analyzed.

For example, either anti-bovine IgG-HRP or specific monoclonal IgG-HRP may be used in arrays printed with antigens and antibodies, respectively. The final detection antibody in antigen arrays will often be anti-bovine IgG-HRP, while for antibody arrays it will often be a HRP conjugated IgG antibody specific for the targeted analyte or a bovine IgG specific for the target analyte that is detected later on the next step with anti-bovine IgG-HRP. These controls can provide a positive control in addition to providing information on the performance or quality of the HRP substrate.

The target protein or antigen and mouse IgG, bovine IgG and anti-bovine IgG present on antigen or antibody arrays can act either as positive or negative controls depending on the array format, in addition to providing information of assay specificity. For example, antigen or mouse IgG spots should provide the positive signal in antibody arrays, while the latter two should provide a positive signal in antigen arrays. These controls can also serve as controls for overall assay performance.

The terms “sample” and “specimen” as used herein are used in their broadest sense to include any composition that is obtained and/or derived from biological or environmental source, as well as sampling devices (e.g., swabs) that are brought into contact with biological or environmental samples. “Biological samples” include body fluids such as milk, urine, blood, plasma, fecal matter, cerebrospinal fluid (CSF), semen, respiratory tract mucus or washing and saliva. In one embodiment, the biological sample is fluid obtained from a mammal, including milk, blood, plasma, serum, and stool. These examples are illustrative and are not to be construed as limiting the sample types applicable to the present invention.

In various aspects of the present invention, the sample is a milk or blood sample, including a plasma or serum sample.

The assay techniques used in conjunction with the substrates of the present invention include any of a number of well-known colorimetric enzyme-linked assays. Examples of such systems are well known in the art. The assay techniques are based upon the formation of a complex between a complementary binding pair, followed by detection with a colorimetric detection system comprising an enzyme-conjugate label and a colorimetric substrate. The detection system will be described with reference to enzyme-linked immunosorbent assays (ELISA). However, a skilled person would appreciate that such techniques are not restricted to the use of antibodies but are equally applicable to any colorimetric assay.

In one embodiment, the ELISA is in the “sandwich” assay format. In this format, the target analyte to be measured is bound between two antibodies—the capture antibody and the detection antibody. In another embodiment, the ELISA is a non-competitive assay in which an antibody binds to the capture antigen and the amount of bound antibody is determined by a secondary detection antibody.

Either monoclonal or polyclonal antibodies may be used as the capture and detection antibodies in sandwich ELISA systems. Monoclonal antibodies have an inherent monospecificity toward a single epitope that allows fine detection and quantitation of small differences in antigen. A polyclonal antibody can also be used as the capture antibody to bind as much of the antigen as possible, followed by the use of a monoclonal antibody as the detecting antibody in the sandwich assay to provide improved specificity. A monoclonal antibody can also be used as the capture antibody to provide specific analyte capture, followed by the use of a polyclonal antibody as the detection antibody in the sandwich assay. Additionally, both the capture and the detection antibodies could be monoclonal.

The term “antibody” as used herein includes naturally occurring antibodies as well as non-naturally occurring antibodies, including, for example, single chain antibodies, chimeric, bifunctional and humanized antibodies, as well as antigen-binding fragments thereof. Such non-naturally occurring antibodies can be constructed using solid phase peptide synthesis, can be produced recombinantly or can be obtained, for example, by screening combinatorial libraries consisting of variable heavy chains and variable light chains (see Huse et al., Science 246: 1275-1281, 1989, which is incorporated herein by reference). These and other methods of making, for example, chimeric, humanized, CDR-grafted, single chain, and bifunctional antibodies are well known (Winter and Harris, Immunol. Today 14: 243-246, 1993; Ward et al., Nature 341: 544-546, 1989; Harlow and Lane, Antibodies: A Laboratory Manual (Cold Spring Harbor Laboratory Press, 1999); Hilyard et al., Protein Engineering: A Practical Approach (IRL Press 1992); Borrabeck, Antibody Engineering, 2d ed. (Oxford University Press 1995); each of which is incorporated herein by reference). In addition, modified or derivatized antibodies, or antigen binding fragments of antibodies, such as pegylated (polyethylene glycol modified) antibodies, can be useful for the present methods. As such, Fab, F(ab′)2, Fd and Fv fragments of an antibody that retain specific binding activity are included within the definition of an antibody.

The term “secondary antibody” refers to an antibody that will bind a target analyte and that is conjugated with either an adaptor molecule such as biotin or an enzyme label such as horseradish peroxidase (HRP). Antibody-adaptor conjugates are processed to give a detectable result by contacting the antibody-adaptor conjugate with an adaptor-enzyme conjugate and then the enzyme substrate; for example, antibody-biotin conjugates will bind streptavidin-HRP conjugates. Antibody-enzyme label conjugates include antibody-HRP conjugates. Use of secondary antibodies is discussed and exemplified below.

The term “binds specifically” or “specific binding activity” or the like, means that two molecules form a complex that is relatively stable under physiologic conditions. The term is also applicable where, an antigen-binding domain is specific for a particular epitope, which is carried by a number of antigens, in which case the antibody carrying the antigen-binding domain will be able to bind to the various antigens carrying the epitope. Specific binding is characterized by a high affinity and a low to moderate capacity. Typically, the binding is considered specific when the affinity constant is about 1×10⁻⁶ M, generally at least about 1×10⁻⁷ M, usually at least about 1×10⁻⁸ M, and particularly at least about 1×10⁻⁹ M or less.

After array manufacture and prior to sample addition, all available protein-binding sites on the substrate surface are blocked by addition and incubation with one or a combination of reagents. These reagents are called “Blockers” and serve to decrease or at best eliminate non-specific protein binding from the sample on the substrate surface, thereby decreasing overall background signal. This increases the ratio of signal to noise, thereby increasing the overall sensitivity of the assay. Blockers play no active part in the subsequent reactions between the sample and other assay reagents and the immobilized proteins on the substrate. Exemplary blockers include, but are not limited to, bovine serum albumin, casein, non-fat dry milk, gelatin derived from fish, pigs and other sources, dextran, serum derived from sources other than the sample being analyzed such as from steelhead salmon, guinea pigs, hamsters, rabbit and other sources, polyethylene glycol, polyvinyl pyrrollidone, and commercial preparations including HeteroBlock (Omega Biologicals, Bozeman, Mont.), SuperBlock, StartingBlock, SEA BLOCK (Pierce, Rockford, Ill.). Typically, blockers are made up in buffer solutions such as, for example, phosphate buffer, phosphate buffered saline, Tris buffer, acetate buffer and others. The blockers may also be supplemented with detergents such as, for example, Tween 20, Tween 80, Nonidet P40, sodium dodecyl sulfate and others.

An important consideration in designing an array is that the capture and detection antibodies of each binding pair must recognize two non-overlapping epitopes so that when the antigen binds to the capture antibody, the epitope recognized by the detection antibody must not be obscured or altered. A large number of complementary binding pairs have already been developed for ELISA and can be used in the present invention.

For multiplexed assays, it is also important that there is no overlap between each of the binding pairs to eliminate cross-reactivity. A number of multiplexed ELISAs have been developed and it is anticipated other combinations of binding pairs could be configured through testing.

In one aspect, the enzyme-conjugate label comprises an enzyme including but not limited to horseradish peroxidase, alkaline phosphatase, ß-D-galactosidase or glucose oxidase.

In an additional aspect, the enzyme label may be conjugated directly to a primary antibody or introduced through a secondary antibody that recognizes the primary antibody. It may also be conjugated to a protein such as streptavidin if the primary antibody is biotin labelled.

In a further aspect, the assay detection system comprises a colorimetric detection substrate comprising 3,3′, 5,5′-tetramethylbenzidine, diaminobenzidine, metal-enhanced diaminobenzidine, 4-chloro-1-naphthol, colloidal gold, nitro-blue tetrazolium chloride, 5-bromo-4-chloro-3′-indolylphosphate p-toluidine salt and naphthol AS-MX phosphate+Fast Red TR Salt.

In certain aspects, the colorimetric reaction can be detected and optionally quantified and analyzed using an image capture device such as a digital camera or a desktop scanner attached to a computer. Known methods for image analysis may be used. For example, the concentration values of known standard elements can be used to generate standard curves. Concentration values for unknown analytes can be analyzed using the standard curve for each analyte to calculate actual concentrations. Values for each analyte can be identified based on the spotting position of each capture element within the array.

The present invention provides methods of in vitro diagnostic applications for the detection of an M. bovis infection such as manual multiplex immunoassay, automated multiplex immunoassay (MIA), Automated MIA (e.g., PictArray™, U.S. Pat. No. 9,625,453), multiple lateral flow immunoassay (LFIA), manual singleplex ELISA (solid phase), automated chemiluminescent immunoassay (CLIA), wash-free immunoassays (manual and automated), automated centrifugal microfluidics-based immunoassay, lab-on-a-chip based immunoassay, paramagnetic bead-based manual ELISA, point-of-care (POC) immunoassays, smart phone based immunoassay and other immunoassay formats. In one aspect, detection is by colorimetric imaging (e.g., PictArray™, U.S. Pat. No. 9,625,453), absorbance (e.g., manual ELISA), chemiluminescence (e.g., automated CLIA, CRET), florescence (e.g., manual immunoassays, ELISA, FRET) and by the naked eye (e.g., lateral flow immunoassays).

In a MIA, anti-M. bovis antibodies (Abs) are determined via an indirect immunoassay (IA). A membrane-free MIA procedure will be used for the printing of microarray spots (e.g., PictArray™, U.S. Pat. No. 9,625,453) directly on the solid surface of a 96-well microtiter plate (MTP). The biorecognition elements responsible for the binding anti-M. bovis antibodies will be printed onto the surface of the MTP well using a leach-proof biomolecular immobilization procedure.

The automated MIA automates all the steps in the manual MIA and employs an integrated colorimetric reader and image analysis software. The automated IAs are ideal for high-throughput tests to detect M. bovis infection in cattle.

For a multiplex LFIA, biomarkers are immobilized in two different lines on a nitrocellulose membrane strip. The results from this assay can be interpreted by naked eye. Alternatively, the rapid LFIA tests could be performed using previously described MIA formats including manual and automated MIAs. The biorecognition elements responsible for the detection of anti-M. bovis Abs would be immobilized in two control lines on a nitrocellulose membrane strip.

Manual singleplex ELISA could also be used to detect an M. bovis infection and would use custom-synthesized M. bovis antigens that would be specific for various anti-M. bovis antibodies.

Automated multiplex and singleplex CLIAs can also be developed for the diagnosis of an M. bovis infection in cattle. The M. bovis antigens could be bound covalently to paramagnetic beads (micron-sub-micron size) and used for the detection of anti-M. bovis Ab in sample. Various formulations of paramagnetic beads could be coated with M. bovis antigens and used for MIA. The detection signal in the case of automated CLIAs could be generated by conjugating the detection antibody with acridinium or other chemiluminescent labels and providing the appropriate trigger solutions for the generation of a chemiluminescent signal.

Manual and automated wash-free MIAs could be developed for the detection of anti-M. bovis Abs. Another wash-free MIA format, such as that based on electrochemiluminescent ELISA, could also be developed. It will involve the detection of analytes in a sample using biomolecule-coated (antibody- or antigen-coated) carbon electrode surface-based microwell plates and SULFO-TAG-labeled detection Ab that emits light upon electrochemical stimulation.

The centrifugal microfluidics-based automated IA could also be performed to detect an M. bovis infection. The M. bovis antigens can be covalently bound to paramagnetic beads and used for the detection of anti-M. bovis antibodies. The magnetic beads are transferred from one chamber to another in the apparatus by a magnet. The detection of analyte occurs in a reaction chamber, which is followed by washing the specific immune complexes formed on paramagnetic beads and their transfer to the detection chamber for the generation and reading of assay signal. The assay signal most commonly used in IA is the chemiluminescent, however, absorbance and fluorescence could also be used. All the microfluidic operations could be performed in a compact benchtop centrifugal microfluidic analyzer.

A lab-on-a-chip (LOC)-based IA could also be developed that could use MIA procedures similar to those previously described. Such LOC-based IAs could use paramagnetic beads or solid surfaces for the covalent attachment of M. bovis antigens. The detection signal could be chemiluminescent, fluorescent, absorbance, electrochemical or colorimetric.

Manual singleplex ELISA can be used to detect an M. bovis infection, employing paramagnetic beads that could be bound covalently to M. bovis antigens.

A point of care (POC) IA, such as that based on electrochemical detection using a disposable strip, can also be developed. The IA would be a label-free immunoassay using an electrochemical reader or a smartphone-based reader. Various smartphone-readout based IAs could also be developed where an optomechanical attachment enables the readout of colorimetric detection signal via the smartphone back camera. Therefore, the smartphone will act as the POC colorimetric readout device.

In addition to the assay formats mentioned above, the invented assay formats for the detection of an M. bovis infection could also be used for the development of novel and emerging IA formats, which are based on advances in complementary technologies. Some examples of such prospective IA formats are wash-free IA based on FRET (fluorescence resonance energy transfer) or CRET (chemiluminescence resonance energy transfer); signal-enhanced IA based on the use of nanoparticle-based signal detection step or the use of micro- and subsmicro- beads for binding capture antibodies/antigens; rapid multiplex IAs based on lab-in-a-tube technology, and vertical microfluidics.

The substrates of the present invention are particularly amenable to use in kits for the detection of target analytes. Such kits may comprise the substrates together with instructions and any assay consumables required. Various kits are envisaged for different target analytes and types of array.

Accordingly, in an additional embodiment, the present invention provides a kit for detecting a plurality of target analytes in a sample, having a substrate having at least two capture elements specific for Mycoplasma bovis (M. bovis) on the substrate, each capture element corresponding to and being able to bind a target analyte, and optionally one or both of a background reducing reagent, and c) a colorimetric detection system. In one aspect, the kit comprising one or more items including a wash solution, one or more antibodies for detection of antigens, ligands or antibodies bound to the capture elements or for detection of the positive controls, software for analyzing captured target analytes, a protocol for measuring the presence of target analytes in samples, a sample diluent, blotting TMB (3,3′,5,5′-tetramethylbenzidine) and/or a secondary antibody. In some aspects, the antibodies for detection are antibody-binding protein conjugates, antibody-enzyme label conjugates, or any combination thereof. In an additional aspect, the sample is milk or a blood sample. In a further aspect, the blood sample is serum or plasma. In one aspect, the substrate is a solid or porous substrate. In certain aspects, the solid substrate is a paramagnetic bead, microtiter plate, or microparticle. In an additional aspect, the colorimetric detection system is HRP-labelled anti-bovine IgG Ab. In specific aspects, the secondary antibody has at least one anti-bovine IgG antibody. In a further aspect, the kit detects a M. bovis infection in a mammal. In certain aspects, the mammal is bovine.

In another aspect, the invention also relates to a method of processing a substrate of the invention. Such a method comprises providing a substrate of the invention as described above, adding at least one sample to the substrate, and processing the substrate such that a detectable result is given by two or more of at least one fiduciary marker, at least one positive colorimetric control, and at least one positive control to monitor assay performance.

In a further embodiment, the present invention provides a method for processing a microarray by providing a substrate having at least two capture elements specific for Mycoplasma bovis (M. bovis) on the substrate, each capture element corresponding to and being able to bind a target analyte; adding at least one sample to the substrate; and processing the substrate such that a detectable result is given by two or more of at least one fiduciary marker, at least one positive colorimetric control, and at least one positive control to monitor assay performance. In one aspect, the sample is milk, serum, or plasma. In certain aspects, the capture element is MilAab (SEQ ID NO: 10), MBOVGP45_310 (SEQ ID NO: 11), MBOVGP45_320 (SEQ ID NO: 12), MBOVGP45_0402 (SEQ ID NO: 13), MBOVGP45_0117 (SEQ ID NO: 14), MBOVGP45_0353 (SEQ ID NO: 15), MBOVGP45_0416 (SEQ ID NO: 16), MBOVGP45_0565 (SEQ ID NO: 17), MBOVGP45_0710-EF (SEQ ID NO: 18), an epitope thereof or a combination thereof. In specific aspects, the capture element is MilAab (SEQ ID NO:10), MBOVPG45_310 (SEQ ID NO:11), MBOVGP45_320 (SEQ ID NO:12), MBOVGP45_0402 (SEQ ID NO:13), and/or MBOVPG45_0117 (SEQ IP NO: 14).

In another embodiment, the present invention provides a method for detecting an analyte in a sample by providing a substrate having at least two capture elements specific for Mycoplasma bovis (M. bovis) on the substrate, each capture element corresponding to and being able to bind a target analyte, adding at least one sample to the substrate, and processing the substrate such that a detectable result is provided. In one aspect, the sample is milk, serum, or plasma. In an additional aspect, the detectable result includes two or more of at least one fiduciary marker, at least one positive colorimetric control, and at least one positive control to detect an analyte in the sample. In certain aspects, the capture element is MilAab (SEQ ID NO: 10), MBOVGP45_310 (SEQ ID NO: 11), MBOVGP45_320 (SEQ ID NO: 12), MBOVGP45_0402 (SEQ ID NO: 13), MBOVGP45_0117 (SEQ ID NO: 14), MBOVGP45_0353 (SEQ ID NO: 15), MBOVGP45_0416 (SEQ ID NO: 16), MBOVGP45_0565 (SEQ ID NO: 17), MBOVGP45_0710-EF (SEQ ID NO: 18) or a combination thereof. In specific aspects, the capture element is MilAab (SEQ ID NO:10), MBOVPG45_310 (SEQ ID NO:11), MBOVGP45_320 (SEQ ID NO:12), MBOVGP45_0402 (SEQ ID NO:13), and/or MBOVPG45_0117 (SEQ IP NO: 14). In a further aspect, detecting the analyte is indicative of a M. bovis infection.

In another aspect, the present invention provides methods for processing a microarray by providing a substrate, adding at least one sample to the substrate, and processing the substrate such that a detectable result is given by two or more of at least one fiduciary marker, at least one positive colorimetric control, and at least one positive control to monitor assay performance.

In one aspect, the step of processing the substrate or microarray comprises a blocking step during which available protein-binding sites on the substrate or microarray are blocked with a blocker, an optional wash step, contacting the substrate or microarray with the sample containing the one or more analytes to be measured, a wash step to remove non-bound material from the substrate or microarray, contacting the substrate or microarray with one or more secondary antibodies that correspond to and will bind one or more target analytes and non-target analyte that is bound to an assay performance control, a wash step, and contacting the substrate or microarray with one or both of an enzyme conjugate or an enzyme substrate to generate a detectable result.

In one embodiment, the present invention provides methods for detecting an analyte in a sample comprising providing a substrate, adding at least one sample to the substrate, and processing the substrate such that a detectable result is provided. In one aspect, the detectable result includes two or more of at least one fiduciary marker, at least one positive colorimetric control, and at least one positive control to detect an analyte in the sample.

In another aspect, the substrate of the invention can be used for the simultaneous detection of at least one target analyte in a sample, and preferably a plurality of different target analytes in a sample, and have utility in diagnostic and screening assays.

Thus, the substrates of the invention provide the advantage that they can be adapted to high throughput (or ultra high throughput) analysis and, therefore, any number of samples (e.g., 96, 1024, 10,000, 100,000, or more) can be examined in parallel, depending on the particular support used. A particular advantage of adapting the substrates to high throughput analysis is that an automated system can be used for adding or removing reagents from one or more of the samples at various times, for adding different reagents to particular samples, or for subjecting the samples to various heating cycles.

For example, the automated system may consist of one or more temperature-controlled chambers and one or more robotic arms mounted on a deck that has platforms configured to hold 96-well plates. The movement of the robotic arms and the temperature in the chambers are controlled by a central computer unit. The array plates are stacked on the deck of the instrument. In one embodiment, the plates containing samples to be analyzed are stacked in a chamber with temperature of 4° C. One robotic arm then sequentially transfers each individual array plate on one platform while the other arm sequentially transfers each individual sample plate on the second platform. A nozzle containing 96 disposable tips then aspirates a predetermined volume of sample from each well of the sample plate and transfers the sample to the corresponding wells of the array plate. The array plate containing the sample is then transferred to a chamber with temperature of 37° C. This process is repeated until a sample has been added to all the array plates stacked on the deck. The array plates are incubated for a predetermined time followed by transfer of each plate to the platform for the addition of wash buffer with the nozzle containing 96 disposable tips. The wash buffer is aspirated after a predetermined time and this wash process is repeated multiple (i.e., two or more) times. Each array plate then receives the secondary antibody followed by transfer to a chamber with a temperature of 37° C. The array plates are incubated for a predetermined time followed by transfer of each plate to the platform for the addition of wash buffer with the nozzle containing 96 disposable tips. The wash buffer is aspirated after a predetermined time and this wash process is repeated multiple (i.e., two or more) times. Each array plate then receives the detection reagent followed by incubation for a predetermined time followed by transfer of each plate to the platform for the addition of wash buffer with the nozzle containing 96 disposable tips. The wash buffer is aspirated after a predetermined time and the plate transferred to the 37° C. chamber for drying. The plates are transferred back to the deck after a predetermined period and manually processed to analyze the data.

In addition to the convenience of examining multiple test agents and/or samples at the same time, such high throughput assays provide a means for examining duplicate, triplicate, or more aliquots of a single sample, thus increasing the validity of the results obtained, and for examining control samples under the same conditions as the test samples, thus providing an internal standard for comparing results from different assays.

In one embodiment, the present invention provides a method for detecting an analyte in a sample comprising providing a substrate, adding at least one sample to the substrate, and processing the substrate such that a detectable result is provided. In one aspect, the detectable result includes two or more of at least one fiduciary marker, at least one positive colorimetric control, and at least one positive control to detect an analyte in the sample.

In one embodiment, the present invention provides an isolated peptide including MBOVGP45_320 (SEQ ID NO:12), MBOVGP45_0402 (SEQ ID NO:13), MBOVPG45_0117 (SEQ IP NO: 14), MBOVGP45_0353 (SEQ ID NO:15), MBOVGP45_0416 (SEQ ID NO:16) or MBOVGP45_0565 (SEQ ID NO:17).

In an additional embodiment, the present invention provides an antibody that binds a peptide including MilAab (SEQ ID NO:10), MBOVPG45_310 (SEQ ID NO:11), MBOVGP45_320 (SEQ ID NO:12), MBOVGP45_0402 (SEQ ID NO:13), MBOVGP45_0117 (SEQ ID NO:14), MBOVGP45_0353 (SEQ ID NO:15), MBOVGP45_0416 (SEQ ID NO:16), MBOVGP45_0565 (SEQ ID NO:17) or MBOVGP45_0710-EF (SEQ ID NO:18). In an additional aspect, the antibody is monoclonal.

The term “antibody,” as used herein, refers to immunoglobulin molecules and immunologically active portions of immunoglobulin molecules, i.e., molecules that contain an antigen binding site that specifically binds an antigen. “Native antibodies” and “intact immunoglobulins”, or the like, are usually heterotetrameric glycoproteins of about 150,000 daltons, composed of two identical light (L) chains and two identical heavy (H) chains. The light chains from any vertebrate species can be assigned to one of two clearly distinct types, called kappa (K) and lambda (2), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called α, δ, ε, γand μ, respectively. The subunit structures and three-dimensional configurations of different classes of immunoglobulins are well known.

Experimentally, antibodies can be cleaved with the proteolytic enzyme papain, which causes each of the heavy chains to break, producing three separate antibody fragments. The two units that consist of a light chain and a fragment of the heavy chain approximately equal in mass to the light chain are called the Fab fragments (i.e., the “antigen binding” fragments). The third unit, consisting of two equal segments of the heavy chain, is called the Fc fragment. The Fc fragment is typically not involved in antigen-antibody binding, but is important in later processes involved in ridding the body of the antigen. “Fv” is the minimum antibody fragment that contains a complete antigen-recognition and -binding site. This region consists of a dimer of one heavy- and one light-chain variable domain in a tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. “Single-chain Fv” or “scFv” antibody fragments comprise the VH and VL domains of antibody, wherein these domains are present in a single polypeptide chain. Preferably, the Fv polypeptide further comprises a polypeptide linker between the VH and VL domains, which enables the scFv to form the desired structure for antigen binding. For a review of scFv, see Pluckthun in The Pharmacology of Monoclonal Antibodies, vol. 113, Rosenburg and Moore eds., Springer-Verlag, New York, pp. 269-315 (1994).

An “antigen”, according to the invention, covers any substance that will elicit an immune response. In particular, an “antigen” relates to any substance, preferably a peptide or protein, that reacts specifically with antibodies. According to the present invention, the term “antigen” comprises any molecule which comprises at least one epitope. Preferably, an antigen in the context of the present invention is a molecule that, optionally after processing, induces an immune reaction. According to the present invention, any suitable antigen may be used, which is a candidate for an immune reaction, wherein the immune reaction is preferably a cellular immune reaction. An antigen is preferably a product that corresponds to or is derived from a naturally occurring antigen.

In a further embodiment, the present invention provides a nucleic acid encoding a peptide including MBOVGP45_320 (SEQ ID NO:12), MBOVGP45_0402 (SEQ ID NO:13), MBOVPG45_0117 (SEQ IP NO: 14), MBOVGP45_0353 (SEQ ID NO:15), MBOVGP45_0416 (SEQ ID NO:16) and/or MBOVGP45_0565 (SEQ ID NO:17).

In one embodiment, the present invention provides a method of detecting a Mycoplasma bovis infection in cattle by adding at least one sample to a substrate having at least two capture elements specific for M. bovis on the substrate, each capture element corresponding to and being able to bind a target analyte; and processing the substrate such that a detectable result is given by two or more of at least one fiduciary marker, at least one positive colorimetric control, and at least one positive control to monitor assay performance, thereby detecting a M. bovis infection. In one aspect, the sample is milk, serum, or plasma. In an additional aspect, the capture element is MilAab (SEQ ID NO:10), MBOVPG45_310 (SEQ ID NO:11), MBOVGP45_320 (SEQ ID NO:12), MBOVGP45_0402 (SEQ ID NO:13), MBOVGP45_0117 (SEQ ID NO:14), MBOVGP45_0353 (SEQ ID NO:15), MBOVGP45_0416 (SEQ ID NO:16), MBOVGP45_0565 (SEQ ID NO:17), MBOVGP45_0710-EF (SEQ ID NO:18), an epitope thereof or a combination thereof. In certain aspects, the capture element is MilAab (SEQ ID NO:10), MBOVPG45_310 (SEQ ID NO:11), MBOVGP45_320 (SEQ ID NO:12), MBOVGP45_0402 (SEQ ID NO:13), and/or MBOVPG45_0117 (SEQ IP NO: 14).

### Nucleic Acid Sequences

| Antigen (SEQ ID NO) | Nucleic Acid Sequence |
|---|---|
| MilAab (SEQ ID | CTGGTTCCGCGTGGATCCATCAAAGACGTGAAATCAGATAACAAAACCCC |
| NO: 1) | GCTGGATCTGCGTGCCAGCTCTCCGAACATCATCAAAGAATCAGAAAAAG |
| | TCAAATACGTGGCGCTGGGCGACTCTATTAGTGCCGGTTTTGATTCGCAGC |
| | TGGATCAAGACTATCCGGGCAATTACGATAAAGAGAAAAACGAAGTGACC |
| | GGTATTTCTTTCCCGACGTACCTGGCATCTTTCATCAAAAACAGTAACGCT |
| | GATAACTCTAAACTGGAAAGTTACACCAACTACGCGAGTTCCGGCACCAC |
| | GCTGGAAGATTGGAAATTCCTGCTGACGGTGGATGACAGCAAACTGAAAC |
| | AACTGGCGGAAAACGACAAAAACAAACTGGCCTACTACAAATCTAAATTC |
| | GGTAAAGATATCATCAAAAAAGCCAAAGAAGTGCGTGAAGAACTGCAGA |
| | AAGCCAATCTGGTTACCATTACGCTGGCGGCCAACGATTTCATGGAACTGG |
| | TCGCCAAAAAACTGAAAAACCCGGAATTCATCAAACAGATCCAAAACATC |
| | AAAGCAAACCCGACCAATGCGATTGCCGGCGTTTCATCGGCTTTCCAGGAT |
| | ACGTTCAAAGAAATGAAAAAACGCATCGATGACTTCAGCGCAGAACTGAA |
| | AAAACATGTGAAAACCGAAAACGTTAACTTCATCGGTTACCCGCTGCCGCT |
| | GCCGCACCTGTTCGTGCTGCTGGATGGCTACCTGTTTAAAAACAATGGTAG |
| | CTCTCTGATCGTCTCACAGATGGCGGTGGATGCCCTGAATAAAAACATTCA |
| | GTCGCAAGTTACCAAAAACAAATTTAACTTCATCAACCCGTACGACTTCTC |
| | GTTCTGGACCGATAATAGCAACATTAATAAACTGTCTCCGACGCTGTTCGA |
| | CATCCATCCGGGCAGTTTTGGTTACAAGAAAATGGCACAAGATGTTTTCAT |
| | CAAACTGATTGTCCCGGAACGTAATAGCGACAAACTGAACGAAAACGGCA |
| | TCAAATGGGATAACAAATACATCTTCACCGATAAAGACTCATTCACCACGC |
| | AGCTGGAATTCGCGAACAAATACGAAACCGTTAAAAAAGTCCTGACGGAT |
| | GACATTAACGGTAACCTGTTTAAAGAAGACGAAATCTTCAAAAAATTCAG |
| | CGATAAACGTAAAAACAAATGGGAATGGTACAAAAAACGCGTGCTGAACA |
| | GTTCCCTGGTTAAAAATATTCTGAACGGTGCAATCGACAGTCTGATTCAGT |
| | TTCCGACCATCAAAGAAGCTGATCTGTCATCGCGCCTGTCCAACTTTTTCA |
| | CGAAAAACAACAACGAAAACCTGAACAAACTGAAAAAATGGCTGGAAGA |
| | AAGCGAATTTGTGCCGAAATTCCTGCTGAGATCTGAACTCGAGTTTCAGGA |
| | AACCTACTGGAACAAAGACAAAAAATCTTCTAGTGAACGCGCGAAACTGT |
| | TCGATCTGATCAACATCTTCAAAAACAACCTGCTGAACGAAGCAAACCTG |
| | ATCTCCCTGATTGCTTCATTTTTCTCCAGCGATCTGATCCAAAAAGAAAAA |
| | GAAGAACTGAAAGAAATCATCGCAGAATTCGCTCAGAACCTGATCATCAA |
| | AAACGTGGACGAAAAAATGCTGAGCGATGCAATCAATAAAGCTATTGGCA |
| | ATAATTCGAGCAAAGCGTTCAAAACCGAAAACCTGGCCAAACTGATCAAA |
| | AGTGTTCTGTCTAGTAAATCCATTAAGAAAACCATTGGCGAACTGTTCATC |
| | AACCTGGCGAACAACAGTAAAATCTATGGTCAGGCGAAATCCGTGGTCGA |
| | CTCGAGCGGCCGCATCGTGACTGACTGA |
| MBOVPG45_0310 | CTGGTTCCGCGTGGATCCGAAAATGGTACAATTAAGGCAAAAGAAGTTGC |
| (SEQ ID NO: 2) | AGAAGTGCATCTTCCAGATAATTTCGATATAAAAAAACTTAATTCTTCTGA |
| | AAAAAGAGTTTTTAAAAAGGCAAATTCTGTAAATTTAAATAGTGTAGATTA |
| | TACAAAAGGAACTACCTTTGAAATACCTGGTGAAAAAGATTACTTTTTAAA |
| | AATTTATTCTTTAACTCCACCATCAATTGATGAAAATACAGTTGTGCCTCTT |
| | GATAAGGATAAAGAAGTTAGTGCTGAAATAAATTTGGAATTTAAAATCAT |
| | TTACAAAGACCCTGTTGAAAACACTGTTGAGTTAGTAGATGGCTGGGAGA |
| | AAAAACTTAGCATTAAAAATGACAAATTTATTTTACATTCGCTTAATTCAG |
| | TTTATAACAGTTTCGAAAGTAACAAAGCCAGTGCTTTTGAATTTAAAAACA |
| | TAGATTGGGACAAATTGAAGGCTAAATATTTTGATGCAGAAATAGTTAAAT |
| | GGAGCGATGGTGATACACCTGTAGTTAAAGTTACTAGTGTTGATCCTTTAA |
| | ATCAGAATGTTAAAGTAGGTGATGAAACTAAAATAAGAATTTCTGGCGTT |
| | GATACACCTGAAAAATTTGTGGGTGGAATTAAAAGCAGTAATCTAGAACA |
| | CAACTATGCATTACTTTCATCTAAATTTGCTGAAGAAGCTTTACCAAAGGG |
| | CTCAAAAGTTAGAGTTTATGGCGATTCTAAAGATGCATACGGAAGAATAG |
| | TTGGCGATGTATTTTTTGGAGACAAATATCAATATCTATATTCAGTTGAAA |
| | TAACTCGTTCTGGATTAACTTTACCATATTATGACAATGTAACTTTGGCATT |
| | TAGTTTTGTAAAAAATCCAGTTTACTGGGAGCATTATGCCTTGCTTCGTTTA |
| | GGTGAAGCATTAGAATATGCTAAAACCAACAAAAAAGGATTCTTTAAAAC |
| | ATTTGCATTGCCATACTGGATTCAATCTAACATTTACAAAATGAAGCCTAA |
| | TGGAAAATGGACATTGCTAGATAAACACTCTCCTACAAATCTGTACAACAT |
| | TGAAGGCATTAATTTAATTGATGATTCATACAAAAATAAATAA |
| MBOVPG45_0320 | CTGGTTCCGCGTGGATCCAATGATCCTAAAAATAATTCTGAAGTTATCATA |
| (SEQ ID NO: 3) | TATCAACGTAATGCTAATTCTGATAAAACGACTGAACAATTTGAAAAATAT |
| | AATCAAGTTAACTTACTTTCAAATATAAAAGATTACTTTGATAAACATGAA |
| | CATTTAAATTTAATATCATATAAGGGAGGGGGCAAGCCTGAGACAGTTGA |
| | ATATAGTTTAATGATGCAAAACAACTATATGAGTAAGTACATCAATTTTGA |
| | TACTGAAGCTTTCAAGGCAATTGTTAAAAAAGAACTAAAACTAAGCGACA |
| | AGTTGCTAAGTAGGCTTAAATTCTCATATGACTATAACAACGTTACACGTG |
| | ACCCAGGAAATAATTATGATGTTCTATTCAAGGTTAAAGTTGGCCTTCCGC |
| | TAGAATCAAATGACAAAAGCAAATATGAAAGTGGCTTATACAGCCAACAA |
| | ATCATAGACTTTAGAATCAAAAATGTAAAAGTCAAAGATAATGATAAACC |
| | TTTTGCTGATGCATTGAAGCCTTATGATCAAAAAATTCAAGCTTTAACTAG |
| | TCAAGACTTTGAAGTCAAGCTAGAAAATATTGATGATGAATTAAAACAAG |
| | CTATTACAAAATATGGCATTCATCAACTAAGTTCGAAGCAGTATGAAAAGT |
| | TATTAAAATTTTCTAGTGCTAAATTAGATGAAATATTAAAAGACAATGACA |
| | AAGAATTAGAAATACCTGTTGGTAGCAAAAAAGAAAAATATACGCTTAAA |
| | TTCAATAAATTCATTGAAGATGTTGTGCTAAATGATGGCTCACTATCAAAT |
| | GCTAAAGCAAAAATTAGAATTGCTGCTTCATTGTTTGACAGCAAGAAAAA |
| | GTCTACTGTTTGGGAATCAGGAAAAACAGTTTATGGCTTATTTACAGTACC |
| | TAAAGAGCAAAAATTAATTAGCGACTTGCAGTTAGCTGAATTAATTGATGT |
| | ACACACAATTGGTAACATTGAGCACAATAGTGATCTGTCAACTTTAACAAA |
| | AGGCAACTTATTTATTAACGTAAAAGATGCAAATATTGAAAAAGTTGAAA |
| | TTGATAAAATTACAACTCAAGACTTTAGAAATGCAACTGTTACTTTAAATG |
| | TTAAACTTAAAAACTCGCAGGAATTAGTTAAAGTTGAAAAACATCTTGGTG |
| | TAGGTAGATACTCATTGCTTTTTGATAAACAATTTACAAAACAAAATATTA |
| | AAGCTCCTGCTTTTGCTACTGAAGGAATAACAACTAAAAACTTACCAAGTA |
| | TTGATAAAACCTTTTTTGGTCACTATAATTCACAACTATTTTCAGGCGGATA |
| | TGCTTCAGCAAGAGCTTTCTATGCTGATAATGTAGTTGAGCCCAAATATGT |
| | GCACATTGGTGAAGACTATATTGCACCAGATTTTCAACCAGTCTTGGCTCC |
| | TTATGACGGACAAATTGTTGCTGCTTATGAACTTACAACTAAGGTGGTGGC |
| | TACTGGTGTTGGTACTGTTGTTGTTATTAAAATTCCTGTTGCTAATTTAGAT |
| | TGGAGTCCAAAAGAAAAAGAAATCTATCTTAATGGCAACAATGAACATAT |
| | TTATATGTCATTCTTGCACTTAGATGCCGGTAAAACCTTAAATAACACTAG |
| | TTTAGGCTGGCAATCAGAAACAGTAACATTGGGCAAGAGAACAATTAAGG |
| | TTGTCCCAACTGTGTCTGCTGATACACCTACTGAGGTCAAAAAAGGCCAAA |
| | TTATAGGCTTTTTGGGAACTCAGGACACTAATGGTGGATGGATGGCACATG |
| | CCCATGTTAACTTATACACAAACAGAAATTTCTGGCTTTCTCCAAATCATTT |
| | TAACAAGGAATCAAAACAATCAAACATTGACAAAAGAGTTAAGGAGTATA |
| | AAAAAACTGATAAGAATAAAAAAATAACATACAGAATTGTCGGCAATGCT |
| | GGTGTTGAAGTTTTTTTAAACCCTAAAGCCGTTCATGAAGTAGATCCAAAA |
| | ACTGGTCTTTTACTAAAAGATGGAAAAGGAAAACCTAAAGCTTTAAAAGA |
| | ACTTCGTACATATGTTGACAACATATCAATGCTAAGCAAGGAACAATCTAA |
| | AGGTTATGCAGATCCTAATCTTGTTTATAAATTAAGAGATTCAAAAACATT |
| | TGCTTTTGGTATAGAAGATTTATTTGAACTTAATAAATAA |
| MBOVPG45_0402 | CTGGTTCCGCGTGGATCCAATAATGAGACCAATGAGCCTATAAAAATTGA |
| (SEQ ID NO: 4) | GAATACAGCAAATAATGAATTGATTGCAAAAGTCAATTCATTATTTAAATT |
| | GCTAAATTCTAGTAGTCAAAAAATCAATGATGACAGCGCAGAAGCAAAAA |
| | CACTTAAGAATAAAATAGAAACTAAAATCAATGACTTAAAGTCATTTGATC |
| | TTAGTACTCTAAGCGCCGATCAATTAAACAATTATATTAATGCATTACAAT |
| | TATTAATAAATCAAGCAAATAACTTTATTAATAAGAATAATTTTGAAAATA |
| | TTAACCACAATAAAATGGAAAATGAAAGCGTCACTCCTAAAATAGACAAA |
| | TTACCTCCTAAGCCTATAAATAAGCCTTCTATTCCATTTGATCCTTCGAATA |
| | TACCAACTTTTCCTAATTTTCCTTTTAATCGCTTTAATAACCAAGACAAGCA |
| | CAAATATCCTGAACATCATACAAAATTTCAAACTGTTGATTCTAAAACTTT |
| | GTATAAAGAGTTATATGACAGAACTTTTTCTCTTAAATATCTAACTAAACT |
| | TTCAGATGGAGGACTCTTAAGCAATGGTTCAGGAACTACATGGTTGCTTGA |
| | TTATCATAAATACACGAATGAAACCAACAAATATAAACTATTTTTCGCTAC |
| | TAATTTACACGTTTTAGCTAAATTTAGTAATTCTTTGGAAAAATCAGTTCAA |
| | GAGAAACTTAACTATTATGACCCAACTAACGACAAAGTTGTTGCTGTTGCT |
| | TTAGGTAAAAGCGCTAATGTAACTAATTTTGACCAAAAAGCCAATAAAAC |
| | AACGTCAAATGGTTATTCTCCTGTTACTTATTTTACTAATAGTAGTGAATTT |
| | ATTAATTATGAAAAATCAAATGTTATAACTCCTATGAATCTAACAAATGCA |
| | ATATCAGAACCGAAATTGGTTTTTGCTGCAGTTGATTTTATGAATGATGAA |
| | GCTATCAAAAATATTCAAAGTGGCTTGAATGAATCGGCAGAACAGTATAA |
| | AAACTATAAAACTAAAAGTGGTGAAATTGAACACTATAAAGCTGCCTATG |
| | ATGATTTTGATAGTAAGAAAAAAGTTCCAATTACAGTTGACTTTGCAGTTT |
| | TTGAAGTTGATGTTGATTTAGAAAAGGCTGATGAAACTTTTAAAAGTTGGA |
| | TTAATGATGCTATTAAAGGTTTAGATGGCTACATAAATAGACTAAATGAAA |
| | CAAACTATCTCCCAAATCAAGATAAAAATATTTCTAAATATATGCAAACTA |
| | CTGACTATGTTTCAGCTTCATATGATAGGAGTAATCAAAACAATTTATGGA |
| | ATGCAAAAGACATTTATATTGCTGGTTACCCTAGTCAAAACAATAATGCTA |
| | CTTGGATGCAAAATAATCCTACCGAAAGAAATTCTGATTCAATAACTTCTT |
| | ACAAAGCTGGTTTAAAAAATAAAGATACTTTTGCATTTGCAACTGGAAGTA |
| | TTGAAGAGAAAATTGGCATTCCATCAAATGCAACAATTAATGATAATTATT |
| | GGAACAGAGTAATGGCAAGTTGGTATGGTTATCAATATAATATTAACTTTT |
| | CATCCCTATATTATGGGGCTTCAGGTTCATTAGCCTATAATGAATTTGGCC |
| | AAATGATAGGTATTTATAATGCTGTTACAGCTAGAGTTGATTATGGAGACT |
| | TGCTTCAGTCAGGCGGAATTGCTCCATTCTTACAATCTAGTGATATTAAAG |
| | CAATTGAAAACACAATTTATGCTTACAATCTTATTGATGGAAGCAATAAAT |
| | CAATTTACCAGAATCAAAAAAATTCATTTAGAGAAAATCTTAAAACACTTT |
| | ATCCTAGTGGCTTTTCTGATGGTTCTAAACAAACAAAATTATTCGATAGCA |
| | TTTAA |
| MBOVPG45_0117 | CTGGTTCCGCGTGGATCCCCGGAATTCCCTATTGCATTATTTGCACCAGTA |
| (SEQ ID NO: 5) | TTATCAGCTTCATGTCGTGAAAAGCAAGATTTTAACCCTAAAGATTTTAAA |
| | GATGTTACATTGTCTTTAGCTTCAGATGGTAAAACTGATTTTGCTAAAAAA |
| | TCTCCATCAGAGTTTGTAAAAGAAAATAAATCATCATATGCAAAAGTGTTA |
| | GAAGTTACCCAAAATGGCAGTAAAACAGACAAATACGGTTATGAGCTAGT |
| | TAGCGTTAGACCTAATGACAACTATGGATATTTATATGTCGAATATAAATT |
| | ATTTGACAAAAATAACAAAAAAAATATAACTCCTAAAAAATTTATTTATTT |
| | ACAAGACTTTAAGTCTAATGTCAAAGAACAATTATCTTCTATCAATTCAAA |
| | TTTATATTTCTCTAAAACTAGGAACCTAGGAACCCCAGTTGATAAAAAAAC |
| | ATTATTAGTAACCGAAATAATAAATGACAAACTTCTTTTAGATTTGCAAAG |
| | TAATAACCTACTATCACAAGCTGGAGTCAAAATCTCTAAGGATCCAAAATT |
| | AGACTTTGGCAATGATAATGAAGGTATCAAAAAAGGTTTCGTCAATTTATC |
| | ATTTACTTACACTGTTGAGCATAACGCAAGGGAAACTGGCAATGACTTTCC |
| | AATAAAACTAGCCAAAAAGATTGAAAATTTTAAAATTGATGGCTTCACTG |
| | GATATGAAAGTTTTGATCCTGATAAGTTCATTGAAAAATCATCAGCCACTG |
| | ATGTAACACTTGAGAAAGTTCAAAAAGATCCAATGTCCTATATTTCTTTAA |
| | AGAATGCTGGAGATACAATTTTAACTGATCTTAGCGTTACTAATGGTGGCA |
| | ACAACCAAGCTACAATTAAATTCAAACTACAAACCAGTATATTTGATTCTG |
| | AAGGCAAAGAAACTAAAGTAACTTTTGATAATGAATATGAATATAAATAC |
| | ACCTGA |
| MBOVPG45_0353 | CTGGTTCCGCGTGGATCCTTAGCTTCTTGGGAAATAACAACTAGCAAGAT |
| (SEQ ID NO: 6) | AAATAACAAAAATGAAAATGATATTTCAATACCTAAAAAAGAACCACTAT |
| | ACAAAAATCATTACATTAATGAAATGTTAAACTTTTACACAAATAACAATG |
| | ATAACCACAAAAAAAATTACATTTCACTCCAAGAAAATAAATCAAATACT |
| | CTTTTTGATGAATTGAAATTTTCATTAACGTACTATCCTATTTTTATTTCTAA |
| | TAGAGCATTAAATAACAGACATCAATATCAAACAATAATTAATAAGGCAA |
| | AAGATGCTATACAAAAGCACTTATCAGCCGATTGGTATTGGACAATTAATA |
| | ATCTTAGCCACTTCTGGTTTATTTTTTCACCATATGGTGATTTGTACAAAAA |
| | AATTGATAAGGAAAAAGAATTATATGAGCAAATTCATAATGAATTAGGTT |
| | CATGGATTGCTCGAATACAAAACAATAATCCCGAAAAGTTATTAAAATTTA |
| | AATTTCCAGAAGCTAAAGAAATTATTAGCAAGTGGGTCGATAAAAATAAG |
| | AACGATTTCAAAAGTTATGATCCAGAAATTTTAAGCAAAAAGTCTATTAGC |
| | AATATAGAATCGTGGTATTTAATTTTTGATTCAAATAAAGCCATAAAAATC |
| | TTGAAATATATTGAAAATGACAAGCCCAAATTGCAAATATTAACTGACTTA |
| | CTAATTTTCAAAAATTCTGAAAATATTGAGGAACAAATTACAAAGATTGAA |
| | AATGAGATAAGTAGTAAGCGCGAATCTGATTTGACAAAAGCTATTGAAAA |
| | AAAGAATGAAGAGGAAATTGATGACAAAAAAGAAGAAAATGATTTAGGG |
| | AAAAATTTGACATCAAGAAATGTGAATAGTAATGCTGACACAGAAACAAT |
| | TGCAAAATTAAAAAAGGAAATTGATCTTTCTAAGATTTTGGAAAGCGTGAT |
| | TAAGCTAAAAGGCGATAAAGAATTCTTTGAATTTCATGCAACACATCAGTA |
| | TAATCAACATTTTGTTGATGCAATTGATACTATAAACAAGAATGAACTGTC |
| | AGTATATAGATTTACTATAAGGAACATTGACAATGAAAATTAG |
| MBOVPG45_0416 | CTGGTTCCGCGTGGATCCTCATCATGGAATAATGAAGATGACATTTTCAAT |
| (SEQ ID NO: 7) | TTAAAAGCAAATACGGAAGTAAAAGCCAGTGACATTTTTTATAAAACCTTT |
| | TTATCGCAATTAAAAGCATATACATTAGAGTCTTTACTCAATAACTTGCAA |
| | AGTAGTATATTGACGCTTAATCTTCCTAATAAAGTTGATGAATTTAAGTTA |
| | AGTAATAATAAAGATGACATTATTTTTAAGTATAAAGGTAAAAGTTACTCA |
| | TTAAAAAATGTTGCAAACAAAATAAATGGCTTTGATTTTAATGAAATTTTA |
| | AGACCTTTTACATATGAAAAAGAAGATGGTAAATTTATTGTAAAAAGAGC |
| | AAAAAATATCAATGACAAGACTGATATAGATATTTTATTTAAATTAAAAAC |
| | GGATGAAAAATTAAACTATTCCAACTTTTTTGAATACAAAAGCATAATTTT |
| | CCAAAATTACTATAAAAAAGGCTTACTAGATGATCTAAGTATTCCTGATTT |
| | ACAATATATGTTGCAAAGTGCTTTTGTTAATAGTAGTACACAATTCCCTAT |
| | GCAAGTAACATCAAATAATACAAGAAGCAAGGCATTTTTTAAGTCTAAATT |
| | CCAACAAGAAATTTTAGAGAAACGACTTTCAAATGAGCTAAAAATTTATA |
| | ATTTTGCTTCTAATGGCATAATTTTCGATCATGTCAAATTTAATAATTTAAA |
| | AATTGATAATGATACTATTAAACTCAATATTGACTTACTTGATTCTAACAA |
| | TAATTCCCTTCTTAGTGATAAGTACAAAAATTTAGAATTTAAATTAACTAA |
| | CTTTTCCAAAGGTCAAAGTGACGTCTATTTTGATTTAAAGACTAAGGCGAA |
| | ACTTACTATTGATAATGATGAAGTTAAGTTCAATGAATTAGTTAATAATCC |
| | TGAAATTAAATTTAAGCCAAATCCATTAACATACAGAACCATTGATGATTT |
| | AATGCACCCTACTAAGACATATGAAATATTTAATTTAAATAATACTGCAAT |
| | GCTACTTAGTGAATTAAAAGATGATATTTTAATATCTAATACTCCAGCCAA |
| | ATTTGACTTTAGAATAGACAAATTTGAAAAAACTAAATTATTAAATAACTC |
| | ATTATCAATTGGTAAATTAGTCATAAATGAATCTAAAACAAAACAAAAGT |
| | ATAATTGGTATTCAATTGATTTTACACCACATAAACACATTTTTTCAAATGG |
| | TTTATATTTAAAAAATGAATTAGGGACAATAAATAAAAATAAAGATTCAT |
| | ACTTTAGTTATTCAGTAAATGATAATAGATTTGATAATAAAGGTAATTTAA |
| | GTGTTCCACAAGGAATTAAAGCAACTGATTTTATTGAGAATTCATTTAATG |
| | ACATTGCAAACTTTTTAATTTATCAAAATAAAGATAACTTATTATTATAA |
| MBOVPG45_0565 | CTGGTTCCGCGTGGATCCGCTTGGGATAATAAGGATAAAGCTCACAAAAA |
| (SEQ ID NO: 8) | TCCTAATAATCAAGATCAATCTGTTAGTGAAATTCAAAAGCTAAAAGATGA |
| | GCTTCAAGATGAAATAACTAAAGCAACTAGCAAGCGTAATCATTATCTTGG |
| | CGACTATACAGACTATGCCTTTAATCAATCTGCTAAGGTAATTCAAGAAGC |
| | TGTTTATGTTCATCATAATGCAAAAACAGTTGAAGAAGTTAAGAAAGCTAT |
| | TGAAAAATTAAAAGCGGGAATCAAAGAAATTGACGCTTACTTTGTACTTTC |
| | AAAAGAAGAAAAACAAAAACTTGCCATAAAAGATTTTAATAAATCACTAT |
| | CAGCTGCCAAGGAAGCTAAAGCAAAATTAACTAAGCCAAGTGCAATTGAA |
| | GTATTAAATGATTCAATTGCTAAAGCTGAATCAAATAAAGAAGGAGACCC |
| | ACACTATTTTACAAACCTGCTAAAATGGACTTATAAATCTAAAAAAGATAT |
| | TGAAGAAGCGAAAAAATTAGAAGAAAAAAGTGATAAGGAACTTTTAAAAT |
| | TATATTTATCTAAAGAATCAAATATACTTAATACCTTTAACAGAGAATACA |
| | ATGCTAGTGGTTCTTTTGACGAGCAAGCAGAAAAACTAAAAGAGATTTTCA |
| | AAAAAGCTAATGAAAACAAAAAAGAAGATTATAGAGATCAAGAAGTTAA |
| | GCTAAGAAAAGAAATTTCTGAGATTTTAGAGAAAATATCAAAAGAAGCTA |
| | TTACTCCTTCTAAAGCTAATGAATTAGCTAAATTAGTTAAAAATGGTGAAT |
| | TAATTATTGGTGACGATTATAAGTACATATTATCAGATGCATTTGCTGACA |
| | ATAAGAAAATCAAAAAAGTTACTCTTGGTAAAAAAATTGAAAAAATAGAA |
| | AGGGGGGCTTTTGATAACTCTGGAGTTGAGACTATAACTTTTAATGAAAAT |
| | TTAAAATCATTAGAAGGGTTTAACAATAAAAAAGTTAAAGAATTAACATT |
| | GCCTAAAAAACTAGAGAAATTTGCTGCCTCTAATAGCACTAAAATTACTAA |
| | GTTACTTCTTCCTAAGACATTAAAATCATTTGCACTAAGCAGCACATTTTTA |
| | GAAGAATTAGAATTTGAACCAGACTTTAAGTTTGACAAAGTAAGCCCTGAT |
| | TCATATTATGAATCAATTGCTATTAACCAGCAATTATTGCCTTCGCTTAATA |
| | AAATATATGTTAGCGATGGCTCTGCTAAGAAAAATCTGCTAGAAAAATTA |
| | AATAAAAATATTACAGAATCAAAAAAAATAAAACTAGGAGAAGATTACAT |
| | TAAGTTCCTAGCATATATTGTTAATGTAAAAATTGTTAAAAAGTTTGCTTC |
| | AATAACTGAAGATAAAAAGAAAAAAATTATTGAAGCACTACTTGAGTCGT |
| | CAATTAAAAAAGAAGAAACTAAAAAAACTAAGTCTGGAATTGAAAAAATT |
| | GTTGCTGATCTAAAGAGTGAAAATTTAAAGGACACATTAGAAAAAGTTCTT |
| | GATTCATATAAGTATGAAAAGATCAGTGCTAAAAATAAATTTGAAGTGTTT |
| | GATGCCGCTATAGAAATGTTAAAAGACATATTGAGAGCATATGGTTTTTAT |
| | TTAAAAACTGAAACTATTAAAAACGATCCTGACCTAAAACAGTATCATCAT |
| | GAAACAGGTGTTACTCCAGGAGAAATTATTAATTATTTTGACTCAGAGAAG |
| | GAAAAATATGAAAATGGTAAAAATGGAAGTGATTCAAACCAATGA |
| MBOVPG45_0710-EF | CTGGTTCCGCGTGGATCCCCGGAATTCCCGGTCGACAAAACGGCTATTGA |
| (SEQ ID NO: 9) | CCTGATTAAAAGCCTGGCGACGACGCAGATTGTCCTGAACCACGAAGGCA |
| | CCATCGGCCAGTTCGTGGCCAACATCTTCAACTACGCATTCGATGCTAAAG |
| | ACGCGGGTTCCATGATTTACGATCAGCTGATCAAATCATTCGGCACCGACA |
| | AACTGACGCAATTCATCAGTAAAGATGAATTTTCCACCCTGTTCAAATCAA |
| | TCTTCGTGAAAAACCATAAAGTTATTGCGTCTATCATGCGTAATGTTACCC |
| | TGACGATGAAACAGGACTTCTACAAATACCACAACGCTAAATCGATCGCG |
| | GATGTCCTGAAAATCTATCTGGATAGCCAAGACAAAATCTACCTGATCAGT |
| | AAAGATTTCTCCGTGCTGCTGGAAAGCTTTCTGAAAGAAGAAACCGTTAAA |
| | AACCTGATTCAGAAACTGCTGTCAACGCAACTGTCGCATTATAAAATCGAT |
| | GCCAACTCTGAACAGAACAAAGCATTTTTCAAAGATCTGCTGGACGAACT |
| | GCCGAGTCTGATTCATGATCTGCACATTATCCCGAACGTGCTGAATGGCAT |
| | GTCGAACGGCCTGAATAAACATAAAAGCCTGACCGACCTGTTTGCTGACAT |
| | TCTGCAGCTGACGGCGAAATCCATCAACTTCGAAGATTTCATCTTCGTGCA |
| | GCGTATCCTGAATAGCTCTACCCTGGAAAAACACTCACAAGTTCTGGGTGA |
| | AAACATTAAGAAAATTGCCGAAGGCGCAACCACGGATGACACCCTGGTTA |
| | ACAAATTCATCGATGACTTCGGTCTGTCTAAACTGCTGACGAGCAAAGGCA |
| | TCTCTGATCATGACGCGAAAGATTTCTTTAAACTGATGCTGAAAAGTGGTA |
| | ATACCAAAGGCGCCCTGGACCTGTTTATCGGTGAACTGTTTAAAAACGTTT |
| | CCAAACTGAAAGATGTCAATTCATGGCCGAAACTGATTTCGACGCTGTTCA |
| | ACAGCGTGAATGAGAAAAAAGCCAAAGAACACATCAAAAAATGGATCGTT |
| | GATGTGGTTAACGCCCGCGACTCCAAACTGTTTAATGTCATGGCAAAAATT |
| | ACCATCGACTCACTGAACAAAGAAGGTTATACGATTCCGGTGGATGAAGA |
| | AAGTGAAAAACTGTCCAAATTCCTGGAATCAATCTCGAAAGCGATCGTTG |
| | GCCAGAACAGCAATGAAACCGGCGTCAACGGTCCGATTCTGGAAATCTTC |
| | AACGATGTTATCGACTCTATCTTCAAAACCCTGAAAGAACTGAAAGATAGT |
| | GATGACGTGCTGACGAAAATTGCCGAAGCCGTTAAAAACGGTGCCCTGAA |
| | ATTTATCTCTGATGGCGACACCATCAGTATCGCAAAAGTTTTCGATAACAT |
| | CCCGAAATTCGAAAAACTGCTGGAAAAAGTCGATCCGAAATCGTATGCTG |
| | ACTTCATCAACGTGATGTTTAAATACGCGCCGAGCGATGAAAACAAAGGT |
| | CTGTTCAACGCTATCTTCAACAGCGATAAATCTAAAGGCAATACCAGCCTG |
| | GGTGCACGTGGTTTCTGGGGCGTCATTCGCGGTAAAGTGCAGCAACTGATC |
| | TCAGCATTTGTCTCGCCGCTGATGAAAAACTACATCGATGAACTGCTGAAA |
| | AGCGAAAAAGTGTACAAAACCGCGCAGGAAGTTAAAGAAAATGTCGAAG |
| | GCTATCAAGCTGTTTGGCGCGTGTATGCCAGCCTGGCCGCAATGCTGTATA |
| | GCAACACGCCGTCTGGTCTGTTCTGGAACGCCACCAATCTGACGTCCGAAG |
| | CATTTCTGCGCGAAGGCTTCCGTCGCGCCTTTCAGGAAGCAATCAAAAACA |
| | AAGATCTGACCAAATACAAAGACAATTACGCAGCTATTGGTCTGATCAAC |
| | GCTAAAGATGTCAATGACAAATTTTGGGCGGGTGTGTCTGGCCTGCGCCAT |
| | AGTTCCATTAGTTATTGGCTGCGTGATCACTATTACGCGCGCGACTACGTG |
| | CTGATCTACATCTACTACAAAGATAGTAAAGACACCAAATACAACGGCCA |
| | GAAAACGAAAAAACAAGTGCTGATTGAAGACCTGAAAAAAGGCTTTATGC |
| | CGGTGGACCCGCAACCGAAATGA |
| MilAab (Native | ATTAAAGATGTGAAAAGTGACAATAAAACTCCTTTAGATTTAAGAGCGTC |
| sequence) (SEQ ID | AAGCCCCAACATTATTAAAGAGTCAGAAAAAGTTAAATATGTTGCATTAG |
| NO: 19) | GTGATTCTATATCAGCCGGTTTTGACTCACAATTAGATCAAGATTACCCAG |
| | GTAATTATGATAAAGAAAAAAACGAAGTAACTGGTATTTCATTCCCTACCT |
| | ATTTAGCTTCATTTATTAAAAATTCAAATGCCGATAATTCTAAATTAGAAT |
| | CATATACAAACTATGCATCTTCAGGCACGACCTTAGAAGATTGAAAATTTT |
| | TACTTACTGTTGATGATTCTAAATTAAAGCAATTAGCAGAAAATGATAAGA |
| | ATAAGCTAGCTTATTATAAGTCTAAATTTGGCAAAGACATAATTAAAAAGG |
| | CCAAAGAAGTAAGAGAAGAATTGCAAAAGGCTAATTTGGTAACTATAACT |
| | TTGGCTGCCAATGACTTTATGGAATTAGTTGCTAAAAAACTAAAAAATCCT |
| | GAATTTATAAAGCAGATACAAAATATCAAAGCCAATCCAACTAATGCAAT |
| | AGCTGGAGTTAGTTCAGCATTTCAAGACACATTCAAAGAAATGAAGAAAA |
| | GAATAGATGACTTTTCTGCTGAGTTAAAAAAACATGTAAAGACTGAAAAT |
| | GTCAATTTTATAGGCTACCCACTGCCACTTCCACATTTATTTGTTTTATTAG |
| | ATGGCTATTTATTTAAGAATAATGGATCATCATTAATTGTTAGTCAAATGG |
| | CTGTTGATGCACTAAATAAAAATATTCAGTCACAAGTTACTAAAAATAAAT |
| | TTAATTTTATTAATCCATATGATTTTTCATTCTGAACAGATAATTCTAATAT |
| | AAACAAATTGTCTCCTACATTATTTGATATTCACCCTGGCAGTTTTGGCTAC |
| | AAAAAAATGGCACAGGATGTGTTTATTAAGCTTATTGTTCCTGAAAGAAAC |
| | AGCGATAAATTAAATGAGAATGGCATAAAGTGAGATAACAAGTACATTTT |
| | TACTGATAAAGATTCATTTACAACTCAATTAGAGTTTGCTAACAAATATGA |
| | GACAGTTAAAAAAGTTTTAACCGATGATATTAATGGAAATCTCTTTAAAGA |
| | AGACGAAATATTCAAAAAGTTTAGCGATAAAAGAAAAAATAAATGAGAAT |
| | GATACAAGAAAAGAGTTTTAAACAGCTCATTAGTTAAAAACATATTAAAT |
| | GGCGCTATTGATTCATTAATTCAATTTCCCACAATAAAGGAAGCCGATCTC |
| | AGTAGCAGATTAAGTAATTTTTTTACGAAAAACAATAATGAAAATCTAAAC |
| | AAATTAAAAAAATGGTTAGAGGAATCAGAATTTGTTCCTAAATTCTTGCTT |
| | AAATCTGAGCTTGAGTTTCAAGAAACTTATTGAAATAAAGACAAAAAGTC |
| | AAGTAGCGAGAGAGCCAAACTTTTCGATTTAATAAACATTTTCAAGAATAA |
| | TCTACTAAATGAAGCTAATTTAATTTCATTAATTGCTTCATTCTTTAGTTCT |
| | GACTTAATTCAAAAAGAAAAAGAAGAATTAAAAGAAATAATAGCGGAATT |
| | TGCACAGAATTTAATTATTAAAAATGTTGATGAAAAAATGTTGTCTGATGC |
| | TATAAATAAAGCAATTGGGAATAACTCATCAAAGGCTTTTAAAACTGAAA |
| | ATCTTGCTAAATTGATTAAATCAGTTTTAAGCAGTAAGAGCATTAAAAAAA |
| | CAATCGGAGAATTATTCATTAACCTTGCAAATAATTCTAAGATTTATGGAC |
| | AAGCTAAATCAGTT |
| MBOVPG45 0710-EF | AAAACTGCAATTGATTTAATTAAATCACTTGCAACAACTCAAATTGTTCTT |
| (native sequence) | AATCATGAAGGTACAATCGGCCAATTTGTAGCGAACATATTTAATTATGCT |
| (SEQ ID NO: 20) | TTTGATGCAAAAGATGCTGGTTCAATGATATATGATCAGCTAATTAAGTCA |
| | TTTGGCACTGACAAATTAACTCAATTTATTTCAAAAGATGAGTTCAGTACT |
| | TTATTCAAGTCAATTTTTGTCAAAAATCACAAAGTTATTGCTTCTATAATGC |
| | GAAATGTAACATTAACAATGAAGCAAGATTTTTATAAATATCACAATGCTA |
| | AATCAATTGCTGATGTTCTTAAAATATATTTAGATAGCCAGGATAAAATTT |
| | ATCTAATTTCTAAAGACTTTTCAGTCTTGCTTGAAAGCTTTTTGAAAGAAG |
| | AAACTGTCAAAAATCTTATTCAAAAATTACTCAGTACACAACTAAGCCACT |
| | ATAAAATTGATGCAAATAGTGAGCAAAACAAAGCATTCTTCAAGGATTTA |
| | TTGGATGAATTGCCTTCATTAATTCATGACTTGCACATTATTCCAAATGTGC |
| | TAAATGGTATGAGTAACGGATTAAATAAACATAAATCATTGACTGATTTAT |
| | TTGCTGATATATTACAACTTACAGCCAAATCTATAAACTTTGAAGACTTTA |
| | TTTTTGTACAGAGAATTTTAAACTCTTCAACATTAGAAAAGCATTCACAAG |
| | TCTTAGGTGAGAATATAAAGAAAATTGCTGAGGGCGCAACTACAGATGAT |
| | ACCTTAGTAAACAAATTTATTGATGATTTTGGTCTTTCTAAGTTATTAACAT |
| | CAAAGGGAATCTCTGATCATGATGCTAAAGATTTCTTTAAATTAATGCTTA |
| | AATCTGGTAACACAAAAGGCGCATTAGACTTATTTATAGGTGAATTATTCA |
| | AAAATGTAAGTAAATTAAAAGATGTTAATTCATGGCCAAAACTAATTTCAA |
| | CATTATTTAATTCTGTTAATGAGAAAAAAGCCAAAGAACACATTAAGAAG |
| | TGAATTGTAGATGTTGTTAACGCAAGAGACAGCAAACTATTTAATGTGATG |
| | GCAAAAATAACAATAGATTCGCTAAATAAAGAAGGCTATACAATTCCTGT |
| | TGATGAAGAATCAGAGAAATTAAGTAAGTTCTTAGAGTCTATTTCAAAGGC |
| | AATAGTTGGCCAAAACAGCAATGAAACAGGCGTAAACGGTCCAATTCTTG |
| | AAATATTTAATGATGTTATAGATAGCATATTTAAAACTCTTAAAGAATTAA |
| | AAGATTCTGATGATGTCCTTACAAAAATTGCAGAAGCAGTTAAAAATGGT |
| | GCCTTAAAGTTCATAAGCGATGGAGATACTATTTCAATTGCTAAGGTATTT |
| | GATAACATTCCTAAGTTTGAAAAACTTTTAGAAAAAGTAGATCCTAAATCA |
| | TATGCTGACTTTATCAATGTTATGTTTAAATATGCACCTTCAGATGAAAAT |
| | AAAGGCCTATTTAATGCAATATTCAACTCAGATAAATCGAAAGGCAATAC |
| | ATCTTTAGGGGCTCGTGGCTTCTGAGGAGTTATAAGAGGTAAGGTTCAACA |
| | GTTAATTTCTGCATTTGTTTCGCCATTAATGAAGAATTATATTGATGAGCTA |
| | TTAAAATCTGAAAAGGTCTATAAAACAGCACAAGAAGTAAAAGAAAATGT |
| | TGAAGGATATCAAGCAGTTTGAAGAGTCTATGCTTCATTAGCTGCAATGTT |
| | ATATTCAAACACACCTAGTGGCTTGTTCTGGAATGCAACTAATTTAACTTC |
| | TGAAGCATTTTTAAGGGAAGGTTTTAGAAGAGCATTTCAAGAGGCAATAA |
| | AAAATAAGGACCTAACTAAATATAAAGATAACTATGCAGCTATAGGACTA |
| | ATTAATGCTAAAGATGTAAATGACAAATTTTGAGCAGGTGTTTCTGGTCTA |
| | CGTCATAGTTCAATTAGTTATTGACTAAGAGACCATTATTATGCACGTGAT |
| | TATGTGTTGATTTATATCTATTACAAAGACTCTAAAGACACAAAATATAAC |
| | GGACAAAAAACCAAAAAGCAAGTTCTTATTGAAGATCTAAAAAAAGGTTT |
| | TATGCCAGTTGACCCACAACCTAAATAA |

### Amino Acid Sequences

| Antigen (SEQ ID NO) | Amino Acid Sequence |
|---|---|
| MilAab (SEQ ID | MVPRGSIKDVKSDNKTPLDLRASSPNIIKESEKVKYVALGDSISAGFDSQLDQD |
| NO: 10) | YPGNYDKEKNEVTGISFPTYLASFIKNSNADNSKLESYTNYASSGTTLEDWKF |
| | LLTVDDSKLKQLAENDKNKLAYYKSKFGKDIIKKAKEVREELQKANLVTITLA |
| | ANDFMELVAKKLKNPEFIKQIQNIKANPTNAIAGVSSAFQDTFKEMKKRIDDFS |
| | AELKKHVKTENVNFIGYPLPLPHLFVLLDGYLFKNNGSSLIVSQMAVDALNKN |
| | IQSQVTKNKFNFINPYDFSFWTDNSNINKLSPTLFDIHPGSFGYKKMAQDVFIK |
| | LIVPERNSDKLNENGIKWDNKYIFTDKDSFTTQLEFANKYETVKKVLTDDING |
| | NLFKEDEIFKKFSDKRKNKWEWYKKRVLNSSLVKNILNGAIDSLIQFPTIKEAD |
| | LSSRLSNFFTKNNNENLNKLKKWLEESEFVPKFLLRSELEFQETYWNKDKKSS |
| | SERAKLFDLINIFKNNLLNEANLISLIASFFSSDLIQKEKEELKEIIAEFAQNLIIKN |
| | VDEKMLSDAINKAIGNNSSKAFKTENLAKLIKSVLSSKSIKKTIGELFINLANNS |
| | KIYGQAKSVVDSSGRIVTD |
| MBOVPG45_0310 | MVPRGSENGTIKAKEVAEVHLPDNFDIKKLNSSEKRVFKKANSVNLNSVDYT |
| (SEQ ID NO: 11) | KGTTFEIPGEKDYFLKIYSLTPPSIDENTVVPLDKDKEVSAEINLEFKIIYKDPVE |
| | NTVELVDGWEKKLSIKNDKFILHSLNSVYNSFESNKASAFEFKNIDWDKLKAK |
| | YFDAEIVKWSDGDTPVVKVTSVDPLNQNVKVGDETKIRISGVDTPEKFVGGIK |
| | SSNLEHNYALLSSKFAEEALPKGSKVRVYGDSKDAYGRIVGDVFFGDKYQYL |
| | YSVEITRSGLTLPYYDNVTLAFSFVKNPVYWEHYALLRLGEALEYAKTNKKG |
| | FFKTFALPYWIQSNIYKMKPNGKWTLLDKHSPTNLYNIEGINLIDDSYKNK |
| MBOVPG45_0320 | MVPRGSNDPKNNSEVIIYQRNANSDKTTEQFEKYNQVNLLSNIKDYFDKHEHL |
| (SEQ ID NO: 12) | NLISYKGGGKPETVEYSLMMQNNYMSKYINFDTEAFKAIVKKELKLSDKLLSR |
| | LKFSYDYNNVTRDPGNNYDVLFKVKVGLPLESNDKSKYESGLYSQQIIDFRIK |
| | NVKVKDNDKPFADALKPYDQKIQALTSQDFEVKLENIDDELKQAITKYGIHQL |
| | SSKQYEKLLKFSSAKLDEILKDNDKELEIPVGSKKEKYTLKFNKFIEDVVLNDG |
| | SLSNAKAKIRIAASLFDSKKKSTVWESGKTVYGLFTVPKEQKLISDLQLAELID |
| | VHTIGNIEHNSDLSTLTKGNLFINVKDANIEKVEIDKITTQDFRNATVTLNVKL |
| | KNSQELVKVEKHLGVGRYSLLFDKQFTKQNIKAPAFATEGITTKNLPSIDKTFF |
| | GHYNSQLFSGGYASARAFYADNVVEPKYVHIGEDYIAPDFQPVLAPYDGQIV |
| | AAYELTTKVVATGVGTVVVIKIPVANLDWSPKEKEIYLNGNNEHIYMSFLHLD |
| | AGKTLNNTSLGWQSETVTLGKRTIKVVPTVSADTPTEVKKGQIIGFLGTQDTN |
| | GGWMAHAHVNLYTNRNFWLSPNHFNKESKQSNIDKRVKEYKKTDKNKKITY |
| | RIVGNAGVEVFLNPKAVHEVDPKTGLLLKDGKGKPKALKELRTYVDNISMLS |
| | KEQSKGYADPNLVYKLRDSKTFAFGIEDLFELNK |
| MBOVPG45_0402 | MVPRGSNNETNEPIKIENTANNELIAKVNSLFKLLNSSSQKINDDSAEAKTLKN |
| (SEQ ID NO: 13) | KIETKINDLKSFDLSTLSADQLNNYINALQLLINQANNFINKNNFENINHNKME |
| | NESVTPKIDKLPPKPINKPSIPFDPSNIPTFPNFPFNRFNNQDKHKYPEHHTKFQT |
| | VDSKTLYKELYDRTFSLKYLTKLSDGGLLSNGSGTTWLLDYHKYTNETNKYK |
| | LFFATNLHVLAKFSNSLEKSVQEKLNYYDPTNDKVVAVALGKSANVTNFDQK |
| | ANKTTSNGYSPVTYFTNSSEFINYEKSNVITPMNLTNAISEPKLVFAAVDFMND |
| | EAIKNIQSGLNESAEQYKNYKTKSGEIEHYKAAYDDFDSKKKVPITVDFAVFE |
| | VDVDLEKADETFKSWINDAIKGLDGYINRLNETNYLPNQDKNISKYMQTTDY |
| | VSASYDRSNQNNLWNAKDIYIAGYPSQNNNATWMQNNPTERNSDSITSYKAG |
| | LKNKDTFAFATGSIEEKIGIPSNATINDNYWNRVMASWYGYQYNINFSSLYYG |
| | ASGSLAYNEFGQMIGIYNAVTARVDYGDLLQSGGIAPFLQSSDIKAIENTIYAY |
| | NLIDGSNKSIYQNQKNSFRENLKTLYPSGFSDGSKQTKLFDS |
| MBOVPG45_0117 | MVPRGSPEFPIALFAPVLSASCREKQDFNPKDFKDVTLSLASDGKTDFAKKSPS |
| (SEQ ID NO: 14) | EFVKENKSSYAKVLEVTQNGSKTDKYGYELVSVRPNDNYGYLYVEYKLFDK |
| | NNKKNITPKKFIYLQDFKSNVKEQLSSINSNLYFSKTRNLGTPVDKKTLLVTEII |
| | NDKLLLDLQSNNLLSQAGVKISKDPKLDFGNDNEGIKKGFVNLSFTYTVEHNA |
| | RETGNDFPIKLAKKIENFKIDGFTGYESFDPDKFIEKSSATDVTLEKVQKDPMS |
| | YISLKNAGDTILTDLSVTNGGNNQATIKFKLQTSIFDSEGKETKVTFDNEYEYK |
| | YT |
| MBOVPG45_0353 | MVPRGSLASWEITTSKINNKNENDISIPKKEPLYKNHYINEMLNFYTNNNDNH |
| (SEQ ID NO: 15) | KKNYISLQENKSNTLFDELKFSLTYYPIFISNRALNNRHQYQTIINKAKDAIQKH |
| | LSADWYWTINNLSHFWFIFSPYGDLYKKIDKEKELYEQIHNELGSWIARIQNN |
| | NPEKLLKFKFPEAKEIISKWVDKNKNDFKSYDPEILSKKSISNIESWYLIFDSNK |
| | AIKILKYIENDKPKLQILTDLLIFKNSENIEEQITKIENEISSKRESDLTKAIEKKN |
| | EEEIDDKKEENDLGKNLTSRNVNSNADETIAKLKKEIDLSKILESVIKLKGDKE |
| | FFEFHATHQYNQHFVDAIDTINKNELSVYRFTIRNIDNEN |
| MBOVPG45_0416 | MVPRGSSSWNNEDDIFNLKANTEVKASDIFYKTFLSQLKAYTLESLLNNLQSSI |
| (SEQ ID NO: 16) | LTLNLPNKVDEFKLSNNKDDIIFKYKGKSYSLKNVANKINGFDFNEILRPFTYE |
| | KEDGKFIVKRAKNINDKTDIDILFKLKTDEKLNYSNFFEYKSIIFQNYYKKGLL |
| | DDLSIPDLQYMLQSAFVNSSTQFPMQVTSNNTRSKAFFKSKFQQEILEKRLSNE |
| | LKIYNFASNGIIFDHVKFNNLKIDNDTIKLNIDLLDSNNNSLLSDKYKNLEFKLT |
| | NFSKGQSDVYFDLKTKAKLTIDNDEVKFNELVNNPEIKFKPNPLTYRTIDDLM |
| | HPTKTYEIFNLNNTAMLLSELKDDILISNTPAKFDFRIDKFEKTKLLNNSLSIGK |
| | LVINESKTKQKYNWYSIDFTPHKHIFSNGLYLKNELGTINKNKDSYFSYSVND |
| | NRFDNKGNLSVPQGIKATDFIENSFNDIANFLIYQNKDNLLL |
| MBOVPG45_0565 | MVPRGSAWDNKDKAHKNPNNQDQSVSEIQKLKDELQDEITKATSKRNHYLG |
| (SEQ ID NO: 17) | DYTDYAFNQSAKVIQEAVYVHHNAKTVEEVKKAIEKLKAGIKEIDAYFVLSK |
| | EEKQKLAIKDENKSLSAAKEAKAKLTKPSAIEVLNDSIAKAESNKEGDPHYFT |
| | NLLKWTYKSKKDIEEAKKLEEKSDKELLKLYLSKESNILNTFNREYNASGSFD |
| | EQAEKLKEIFKKANENKKEDYRDQEVKLRKEISEILEKISKEAITPSKANELAK |
| | LVKNGELIIGDDYKYILSDAFADNKKIKKVTLGKKIEKIERGAFDNSGVETITFN |
| | ENLKSLEGFNNKKVKELTLPKKLEKFAASNSTKITKLLLPKTLKSFALSSTFLEE |
| | LEFEPDFKFDKVSPDSYYESIAINQQLLPSLNKIYVSDGSAKKNLLEKLNKNITE |
| | SKKIKLGEDYIKFLAYIVNVKIVKKFASITEDKKKKIIEALLESSIKKEETKKTKS |
| | GIEKIVADLKSENLKDTLEKVLDSYKYEKISAKNKFEVFDAAIEMLKDILRAYG |
| | FYLKTETIKNDPDLKQYHHETGVTPGEIINYFDSEKEKYENGKNGSDSN |
| MBOVPG45_0710-EF | MVPRGSPEFPVDKTAIDLIKSLATTQIVLNHEGTIGQFVANIFNYAFDAKDAGS |
| (SEQ ID NO: 18) | MIYDQLIKSFGTDKLTQFISKDEFSTLFKSIFVKNHKVIASIMRNVTLTMKQDFY |
| | KYHNAKSIADVLKIYLDSQDKIYLISKDFSVLLESFLKEETVKNLIQKLLSTQLS |
| | HYKIDANSEQNKAFFKDLLDELPSLIHDLHIIPNVLNGMSNGLNKHKSLTDLFA |
| | DILQLTAKSINFEDFIFVQRILNSSTLEKHSQVLGENIKKIAEGATTDDTLVNKFI |
| | DDFGLSKLLTSKGISDHDAKDFFKLMLKSGNTKGALDLFIGELFKNVSKLKDV |
| | NSWPKLISTLFNSVNEKKAKEHIKKWIVDVVNARDSKLFNVMAKITIDSLNKE |
| | GYTIPVDEESEKLSKFLESISKAIVGQNSNETGVNGPILEIFNDVIDSIFKTLKEL |
| | KDSDDVLTKIAEAVKNGALKFISDGDTISIAKVFDNIPKFEKLLEKVDPKSYAD |
| | FINVMFKYAPSDENKGLFNAIFNSDKSKGNTSLGARGFWGVIRGKVQQLISAF |
| | VSPLMKNYIDELLKSEKVYKTAQEVKENVEGYQAVWRVYASLAAMLYSNTP |
| | SGLFWNATNLTSEAFLREGFRRAFQEAIKNKDLTKYKDNYAAIGLINAKDVN |
| | DKFWAGVSGLRHSSISYWLRDHYYARDYVLIYIYYKDSKDTKYNGQKTKKQ |
| | VLIEDLKKGFMPVDPQPK |

The following examples are provided to further illustrate the embodiments of the present invention, but are not intended to limit the scope of the invention. While they are typical of those that might be used, other procedures, methodologies, or techniques known to those skilled in the art may alternatively be used

### EXAMPLES

### Example 1

Expression of Recombinant M. bovis Antigens

The M. bovis antigens to be used in the multiplex immunoassay (MIA) are recombinant proteins that were generated based on predicted lipoprotein genes from the M. bovis type strain PG45 (NCBI-RefSeq:NC_014760).

TABLE 1
M. bovis PG45 gene sequences corresponding to target antigens

| Antigen | Gene |
|---|---|
| MilAab | MBOVPG45_RS03500 A 605 aa portion from N- |
| | termini (57aa-662aa) |
| 0117 | MBOVPG45_RS00575 |
| 0353 | MBOVPG45_RS01765 |
| 0416 | MBOVPG45_RS02080 |
| 0565 | MBOVPG45_RS02800 |
| 0710-EF | MBOVPG45_RS03500 670 aa C-terminal end |
| 0310 | MBOVPG45_RS01550 |
| 0320 | MBOVPG45_RS01600 |
| 0402 | MBOVPG45_RS02010 |

To facilitate expression of the genes in E. coli, the DNA sequences were modified by altering TGA codons to TGG. A putative signal peptide cleavage site was identified using SignalP, and sequences upstream of and including the cysteine of this predicted lipoprotein cleavage site were removed or altered to ensure that the sequence no longer encoded a cysteine residue. A BamHI (ggatcc) site was introduced at the 5′ end, and two additional stop codons along with a SalI site were introduced at the 3′ end (TGATAgtcgac) to enable cloning of the gene into expression vectors.

The modified gene sequences were either generated by overlap extension PCR mutagenesis or synthesized and provided in a pUC57 construct (Genscript). The constructs were digested with BamHI and SalI (New England Biolabs), the gene inserts purified from an agarose gel using a QiaexII kit (QIAGEN) and ligated into BamHI/SalI digested pGEX-4T-1 (Amersham Pharmacia Biotech) according to the manufacturer's instructions. E. coli JM109 cells were transformed with the ligation mixture, and clones were selected by PCR screening. The DNA sequences of the inserts in the expression vector pGEX-4T-1 were confirmed using BigDye terminator cycle sequencing reaction kit. Recombinant proteins were purified by gravity-flow purification using GST GraviTrap columns (GE Healthcare) following the manufacturer's instructions.

Purified recombinant proteins were visualized on 4-20% Mini-PROTEAN TGX Stain-Free Precast Gels (BioRad) and transferred to Immobilon-P PVDF membrane using the Trans-Blot Turbo Transfer System (BioRad). Western blot membranes were blocked in 5% skim milk, washed in phosphate-buffered saline with 0.1% Tween 20 (PBS-T), and probed with sera (1:100 dilution) or milk (1:2 dilution) in 1% skim milk in PBS-T. HRP-conjugated sheep anti-bovine IgG heavy and light chain antibody (Bethyl) diluted 1:2000 in 1% skim milk in PBS-T was used to detect antibodies from the sera and milk that had bound to the recombinant proteins. The binding was detected by chemiluminescence using the Clarity Western ECL Substrate (BioRad).

TABLE 2
Conditions used for expression of recombinant proteins in E. coli

| | Expected | | | | | | |
|---|---|---|---|---|---|---|---|
| | size of | | | | | | |
| | recom- | | | | | | Induc- |
| | binant | E. | | Cell | IPTG | | tion |
| | protein | coli | Me- | density | conc. | Temp. | time |
| Antigen | (kDa) | strain | dium | (OD₆₀₀) | (mM) | (° C.) | (h) |
| MilAab | 93 | This antigen was provided privately | | | | | |
| 0117 | 60 | JM109 | LB | 0.7 | 0.5 | 37 | 3 |
| 0353 | 69 | JM109 | LB | 0.7 | 0.5 | 37 | 2.5 |
| 0416 | 72 | JM109 | LB | 0.6 | 1.0 | 37 | 2.5 |
| 0565 | 96 | BL21 | YTA | 1.0 | 0.5 | 37 | 2 |
| 0710- | 103 | JM109 | LB | 0.6 | 1.0 | 30 | 3 |
| EF | | | | | | | |
| 0310 | 68 | JM109 | LB | 0.7 | 0.1 | 37 | 3 |
| 0320 | 107 | BL21 | YTA | 0.9 | 0.5 | 37 | 2.5 |
| 0402 | 97 | JM109 | LB | 0.6 | 0.1 | 37 | 3 |
| GST | 26 | JM109 | LB | 0.6 | 0.1 | 37 | 3 |

### Example 2

Membrane-Free M. bovis MIA

The membrane-free MIA format involves the simultaneous detection of antibodies towards different recombinant M. bovis antigens in milk or serum samples, which act as the diagnostic biomarker of M. bovis infection in cattle.

The membrane-free MIA employs the polystyrene flat-bottom 96-well microtiter plate (MTP) as the substrate.

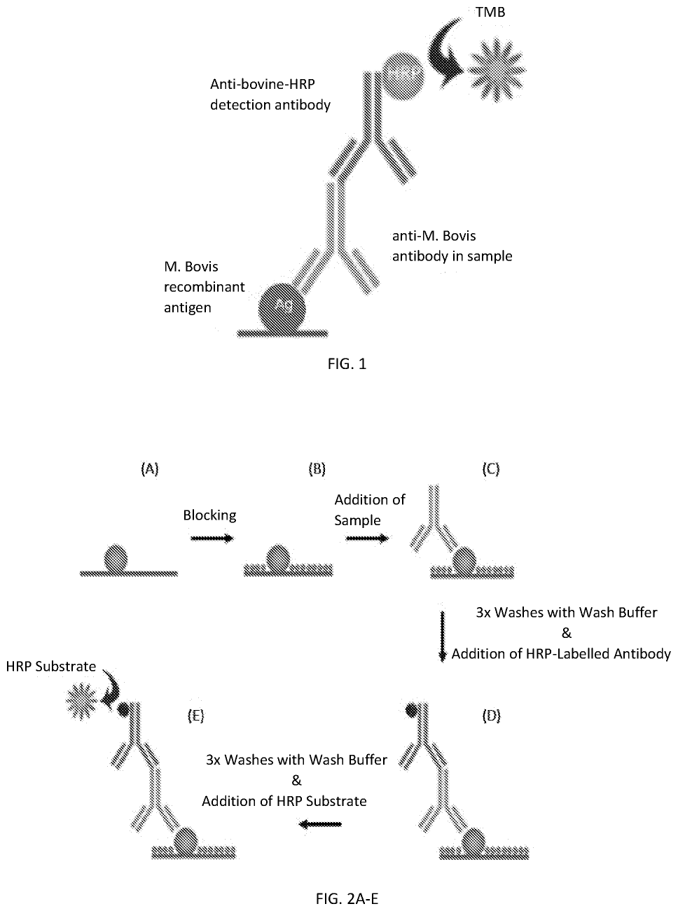

The generalized MIA format of membrane-free M. bovis MIA is shown in a schematic (FIG. 1). The anti-M. bovis antibodies (Ab) would be detected by an indirect IA. The multiple M. bovis recombinant antigens will be printed as spots onto the surface of a MTP's well using a microarray printer. The subsequent steps in the MIA leads to the formation of colored spots if the anti-M. bovis Ab are present in the sample (FIG. 2). The intensities of colored spots are directly proportional to the concentration of anti-M. bovis Abs present in the sample. The colorimetric arrays are then imaged using a colorimetric reader for membrane-free MIA.

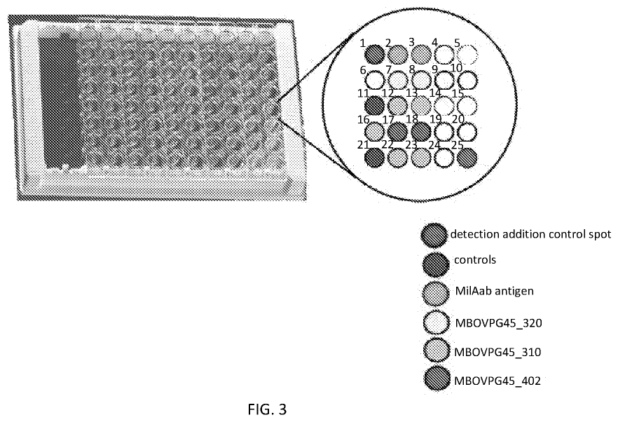

The proof-of-concept of MIA was demonstrated on the detachable 8-well module of a 96-well MTP, where four tests, i.e. MilAab, MBOVPG45_310, MBOVPG45_320, MBOVPG45_0402 and MBOVPG45_0117 were performed into a single MTP's well that also incorporated four IA controls for monitoring the performance of MIA (FIG. 3). The MIA could detect up to nine analytes in duplicate in a single well.

The M. bovis recombinant antigens are printed in the carbonate buffer as the print buffer.

### Example 3

### Printing of Biomolecules on the Solid Substrate

The biomolecules, i.e. M. bovis antigens and controls, were prepared in their specific carbonate buffer as the print buffer. The biomolecules were then printed onto the membrane-free substrate in the desired format, as shown in FIG. 3. Subsequently, the printed 8-well detachable strips of a 96-well MTP are stored overnight and then blocked by incubating with 200 μl of 2.5% casein blocking solution for 1 hour at 37° C. The excess unbound reagents were then washed away in three consecutive washings with Pictor's 1× washing solution.

### Example 4

Protocol for Membrane Free M. bovis MIA

The general protocol is shown in FIG. 2.

Sample: The milk sample is diluted 1:20 using a diluent (PBS-T). 100 μl of the diluted milk sample is added to each MTP's well. The MTP is incubated at 37° C. for 30 min and subsequently washed 3 times with 1× washing solution.

Detection solution: The HRP-labeled secondary Abs are then provided to detect the formation of specific immune complexes on the printed spots. 100 μl of anti-bovine IgG conjugate is added to each MTP's well. The MTP is incubated at 37° C. for 30 min and then washed 3 times with Pictor's 1× washing solution.

TMB: The blotting TMB is used as a substrate for HRP to develop the colorimetric spots. 100 μl of TMB is added to the MTP's well and incubated for 15 minutes at room temperature, where the MTP is covered to protect it from light. Afterward, the liquid is discarded, and MTP is inverted and tapped onto an absorbent paper towel to eliminate any remaining liquid.

Readout: MTP is placed into a commercial reader for the determination of colorimetric intensities of spots. The results of the MIA are generated by the software.

### Example 5

### Colorimetric Readout

The Bovine MIA results in the formation of colorimetric spots, the intensity of which is directly proportional to the concentration of analytes, anti-M. bovis IgG, present in the milk sample. The colorimetric arrays are imaged using a handheld colorimetric reader device or commercially available colorimetric microarray readers.

### Example 6

### Image Analysis

Image analysis software will be used that employs an image analysis algorithm. The software will analyze the colorimetric images of microarray spots in each well, which are captured by commercial readers and generate the desired results. The software will identify the array wells followed by the detection of positive control spots within each well. The positive control spots act as alignment anchors and are used by the software to place a microarray grid for all spots in a well. The image analysis software then determines the pixel intensity for each colorimetric spot based on the image analysis algorithm. The data generated from each spot is then collated.

### Example 7

Automated M. bovis MIA (Membrane-Free, 96-Well MTP)

The automated MIA will be performed inside an analyzer, where all the steps in the manual MIA will be automated. The dispensing and aspiration of reagents will be done by a needle attached to the robotic arm. The assay components will be provided in the form of ready-to-use assay cartridges that are simply plugged inside the analyzer and can perform up to 100 MIA tests. The analyzer would have a dedicated compartment for putting the patient sample vials, and dedicated spaces for placing the wash buffer, TMB substrate and other buffers. The washing of the MTP wells will be done by the robotic needle using specific washing programs. Similarly, the needle will be washed after each dispensing step. However, disposable tips could also be used, which would obviate the cleaning of the needle after each dispensing step. All the steps of the IA will be optimized for the automated MIA. The readout of the colorimetric spots in the processed 96-well MTP will be performed using an integrated colorimetric reader and an image analysis software. The analyzer would need to undergo daily, weekly and monthly maintenance.

### Example 8

### Automated Chemiluminescent Immunoassay (CLIA)

The assay formats used for the development of MIA could be further employed for the development of automated CLIAs, both multiplex as well as singleplex, for the diagnosis of M. bovis in cattle. The M. bovis antigen could be bound covalently to paramagnetic beads (micron-sub-micron size) and used for the detection of bovine IgG against M. bovis via indirect immunoassay. The detection signal in the case of automated CLIAs could be generated by conjugating the detection Ab with acridinium or other chemiluminescent labels and providing the appropriate trigger solutions for the generation of chemiluminescent signal. All the automated CLIAs are performed using a high-throughput analyzer. The assay reagents are stored in the form of assay cartridges that can be used for up to 100 tests. The buffers, wash solution and trigger solutions are stored at the respective places in the analyzer.

### Example 9

### M. bovis ELISA

Manual ELISA can be developed for the detection of M. bovis IgG using the developed MIA procedure with customization of some steps for ELISA. The 96-well MTP would be coated with a mixture of M. bovis antigens either passively or using a leach-proof biomolecular immobilization procedure based on silane chemistry. All the immunoassay steps for the detection of anti-M. bovis IgG by ELISA would then be performed exactly as specified in the bovine MIA except the last step. In the case of ELISA, the signal would be generated by enzyme-substrate reaction by providing TMB and H₂O₂ to the HRP-labeled detection Ab. The enzyme-substrate reaction is stopped by providing a stop solution comprising of 1N H₂SO₄. The optical density of the colorimetric solution is then read at 450 nm with reference at 650 nm.

### Example 10

### Rapid One Step Kinetics-Based ELISA

A customized rapid one step kinetics-based ELISA procedure will be used for the detection of anti- M. bovis IgG, as described by Vashist et al. in Biosensors and Bioelectronics 67, 73-78, 2015.

### Example 11

### Rapid One Step Kinetics-Based ELISA Using Paramagnetic Beads

A customized rapid one step kinetics-based ELISA procedure using paramagnetic beads will be used for the detection of anti-M. bovis IgG, as described by Vashist et al. in Analytical Biochemistry 456, 32-37, 2014.

### Example 12

### Centrifugation Microfluidics-Based Automated Point of Care Immunoassay

A customized centrifugal microfluidics-based automated point-of-care immunoassay procedure using paramagnetic beads will be used for the detection of anti-M. bovis IgG, as described by Czilwik et al. in RSC Advances 5(76), 61906-61912, 2015.

### Example 13

### Wash-Free Immunoassay

Manual and automated wash-free MIAs could be developed for the detection of anti-M. bovis IgG in samples based on electrochemiluminescent ELISA. It will involve the detection of analytes in sample using biomolecule-coated (antibody- or antigen-coated) carbon electrode surface-based microwell plates and SULFO-TAG-labeled detection Ab that emits light upon electrochemical stimulation.

Although the invention has been described with reference to the above examples, it will be understood that modifications and variations are encompassed within the spirit and scope of the invention. Accordingly, the invention is limited only by the following claims.

1. A substrate comprising at least two capture elements specific for Mycoplasma bovis (M. bovis) on the substrate, each capture element corresponding to and being able to bind a target analyte,
the substrate further optionally comprising a plurality of control elements comprising: a) at least one fiduciary marker, b) at least one negative control to monitor background signal, c) at least one negative control to monitor assay specificity, d) at least one positive colorimetric control, e) at least one positive control to monitor assay performance and any combination thereof.

2. The substrate of claim 1, wherein the capture elements bind target analytes, wherein the target analytes are indicative of an M. bovis infection.

3. The substrate of claim 1, wherein the capture element is selected from the group consisting of a protein, lipoprotein, glycoprotein, a protein fragment, a peptide, a polypeptide, a polypeptide fragment, antigen, an antigen fragment, an antigenic determinant, an epitope, a hapten, an immunogen, an immunogen fragment, or any combination thereof.

4. The substrate of claim 1, wherein the capture element is selected from the group consisting of MilAab (SEQ ID NO: 10), MBOVGP45_310 (SEQ ID NO: 11), MBOVGP45_320 (SEQ ID NO: 12), MBOVGP45_0402 (SEQ ID NO: 13), MBOVGP45_0117 (SEQ ID NO: 14), MBOVGP45_0353 (SEQ ID NO: 15), MBOVGP45_0416 (SEQ ID NO: 16), MBOVGP45_0565 (SEQ ID NO: 17), MBOVGP45_0710-EF (SEQ ID NO: 18), an epitope thereof and a combination thereof.

5. The substrate of claim 1, wherein the capture element comprises MilAab (SEQ ID NO:10), MBOVPG45_310 (SEQ ID NO:11), MBOVGP45_320 (SEQ ID NO:12), MBOVGP45_0402 (SEQ ID NO:13) and MBOVGP45_0117 (SEQ ID NO:14).

6. The substrate of claim 1, wherein the target analyte is an antibody or an antibody fragment.

7. The substrate of claim 1, wherein the substrate is a solid or porous substrate.

8. The substrate of claim 7, wherein the solid substrate is a paramagnetic bead, microtiter plate, microparticle, or magnetic bead.

9. A kit for detecting a plurality of target analytes in a sample, comprising a) a substrate of claim 1 and optionally one or both of b) a background reducing reagent, and c) a colorimetric detection system.

10. The kit of claim 9, further comprising one or more items selected from the group consisting of: a) a wash solution, b) one or more antibodies for detection of antigens, ligands or antibodies bound to the capture elements or for detection of the positive controls, c) software for analyzing captured target analytes, d) a protocol for measuring the presence of target analytes in samples, e) a sample diluent, f) blotting TMB (3,3′,5,5′-tetrarnethylbenzidine) and g) a secondary antibody.

11-15. (canceled)

16. The kit of claim 9, wherein the colorimetric detection system comprises HRP-labelled anti-bovine IgG Ab.

17. The kit of claim 9, wherein the secondary antibody comprises at least one anti-bovine IgG antibody.

18. (canceled)

19. The kit of claim 18, wherein the mammal is bovine.

20. A method for processing a microarray comprising:
a) providing a substrate of claim 1;
b) adding at least one sample to the substrate; and
c) processing the substrate such that a detectable result is given by two or more of i) at least one fiduciary marker, ii) at least one positive colorimetric control, and iii) at least one positive control to monitor assay performance.

21-23. (canceled)

24. A method for detecting an analyte in a sample comprising a substrate of claim 1, adding at least one sample to the substrate, and processing the substrate such that a detectable result is provided.

25. (canceled)

26. The method of claim 24, wherein the detectable result includes two or more of at least one fiduciary marker, at least one positive colorimetric control, and at least one positive control to detect an analyte in the sample.

27-29. (canceled)

30. An isolated peptide selected from the group consisting of MilAab (SEQ ID NO: 10), MBOVGP45_310 (SEQ ID NO: 11),-MBOVGP45_320 (SEQ ID NO: 12), MBOVGP45_0402 (SEQ ID NO: 13), MBOVGP45_0117 (SEQ ID NO:14), MBOVGP45_0353 (SEQ ID NO: 15), MBOVGP45_0416 (SEQ ID NO: 16), MBOVGP45_0565 (SEQ ID NO: 17) or MBOVGP45_0710-EF (SEQ ID NO: 18).

31. An antibody that binds any one of the isolated peptides of claim 30.

32. (canceled)

33. A nucleic acid encoding a peptide selected from the group consisting of MBOVGP45_320 (SEQ ID NO: 12), MBOVGP45_0402 (SEQ ID NO: 13), MBOVGP45_0117 (SEQ ID NO:14), MBOVGP45_0353 (SEQ ID NO: 15), MBOVGP45_0416 (SEQ ID NO: 16) or MBOVGP45_0565 (SEQ ID NO: 17).

34. A method of detecting a Mycoplasma bovis infection in bovine comprising:
a) adding at least one sample to the substrate of claim 1; and
b) processing the substrate such that a detectable result is given by two or more of i) at least one fiduciary marker, ii) at least one positive colorimetric control, and iii) at least one positive control to monitor assay performance, thereby detecting a Mycoplasma bovis infection.

35-36. (canceled)
